(19) United States
(12) Patent Application Publication
(10) Pub. No.: US 2016/0311934 A1
(43) Pub. Date: Oct. 27, 2016

(54) HYDROXYALKYL STARCH DERIVATIVES AS REACTANTS FOR COUPLING TO THIOL GROUPS

(71) Applicant: FRESENIUS KABI DEUTSCHLAND GMBH, Bad Homburg (DE)

(72) Inventors: Norbert ZANDER, Bad Nauheim (DE); Sabrina HUMMEL, Gelnhausen (DE); Ulrike WEDEMEYER, Franfurt (DE); Thomas HEY, Bad Nauheim (DE); Dominik HECKMANN, Friedberg (DE)

(21) Appl. No.: 14/777,908

(22) PCT Filed: Mar. 20, 2014
(86) PCT No.: PCT/EP2014/055596

§ 371 Date: Sep. 17, 2015

(30) Foreign Application Priority Data
Mar. 20, 2013 (EP) 13160267.4

(51) Int. Cl. C08B 31/12; A61K 38/17; C12N 9/90; A61K 38/52; A61K 38/38; C12N 9/96; A61K 47/48; A61K 38/57

(52) U.S. Cl. CPC C08B 31/125; A61K 47/4823; A61K 38/1709; A61K 38/1793; A61K 38/57; A61K 38/52; A61K 38/385; C12N 9/96; C12N 9/90; C12Y 502/01008

(57) ABSTRACT

The present invention relates to a hydroxyalkyl starch (HAS) derivative of formula (I) wherein F1 is a functional group comprising the group —NR′—, with R′ being H or alkyl; L is a spacer bridging F1 and S; wherein HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6.

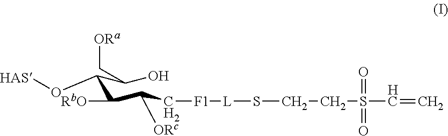

The present invention relates to a hydroxyalkyl starch derivative comprising a vinylsulfone group as well as to a method for preparing the same. Further, the invention relates to the use of said hydroxyalkyl starch derivative as reactant for coupling to a thiol group of a further compound. Further, the present invention relates to a hydroxyalkyl starch derivative coupled to a thiol group of a further compound and a method for preparing the same.

Hydroxyalkyl starch (HAS), in particular hydroxyethyl starch (HES), is a substituted derivative of the naturally occurring carbohydrate polymer amylopectin, which is present in corn starch at a concentration of up to 95% by weight, and is degraded by alpha amylases in the body. HES in particular exhibits advantageous biological properties and is used as a blood volume replacement agent and in hemodilution therapy in clinics (Westphal et al., Anesthesiology, 2009, 111: 187-202). Amylopectin consists of glucose moieties, wherein in the main chain alpha-1,4-glycosidic bonds are present and at the branching sites alpha-1,6-glycosidic bonds are found. The physico-chemical properties of this molecule are mainly determined by the type of glycosidic bonds. Due to the nicked alpha-1,4-glycosidic bond, helical structures with about six glucose-monomers per turn are produced. The physico-chemical as well as the biochemical properties of the polymer can be modified via substitution. The introduction of a hydroxyethyl group can be achieved via alkaline hydroxyethylation. By adapting the reaction conditions it is possible to exploit the different reactivity of the respective hydroxy group in the unsubstituted glucose monomer with respect to a hydroxyethylation. Owing to this fact, the skilled person is able to influence the substitution pattern to a limited extent.

It is generally accepted that the stability of polypeptides can be improved and the immune response against these polypeptides is reduced when the polypeptides are coupled to polymeric molecules, i.e. when a conjugate of the polypeptide with the polymeric molecule is formed. Further, polymeric prodrugs, thus drugs coupled to polymeric compounds, were suggested to prolong the circulation lifetime in the body due to the increase in size of the drug-polymer conjugate when compared to the single drug which may prevent a quick removal of the drug by glomerular filtration through the kidneys.

Some ways of producing a hydroxyalkyl starch derivative for coupling to thiol groups of further compounds, such as cysteine groups of proteins are described in the art.

For example, WO 02/080979 discloses a method for the preparation of hydroxyalkyl starch derivatives for coupling to thiol groups of DNA, wherein a hydroxyalkyl starch is first oxidized at its reducing end, subsequently modified with an amino group and finally reacted with succinimidyl-4-(N-maleimidomethyl)cyclohexane-1-carboxylate (SMCC) to give a maleimide modified HAS derivative. Said HAS derivative is further coupled to a thiol group of DNA resulting in a hydroxyalkyl starch DNA conjugate. Vinylsulfone modified HAS derivatives and their coupling to further compounds are not mentioned.

Similarly, W O 2005/014050 discloses a method for the preparation of hydroxyalkyl starch G-CSF conjugates, wherein e.g. a HAS derivative comprising a maleimide group is disclosed. In this method, the hydroxyalkyl starch is first oxidized at its reducing end, subsequently modified with an amino group and finally reacted with N-alpha(maleimidoacetoxy)succinimide ester (AMAS) to give the maleimide modified HAS derivative. Said HAS derivative is further coupled to a thiol group of G-CSF. Vinylsulfone modified HAS derivatives and their coupling to further compounds are also not mentioned.

Halogenacetyl modified HAS molecules and their coupling to thiol groups of further compounds are further described e.g. in WO2003/070772 and EP 1 398 322 A1. In the described method, HAS is modified via its oxidized reducing end with a linker compound to give the halogenacetyl modified HAS derivate.

Many of the above-described conjugation methods employ a type of chemistry whereby activated carboxylic acid derivatives of hydroxyalkyl starch are either formed as intermediate products or are used to introduce a functional group into the hydroxyalkyl group via a linker. Such chemistry is also described in WO 2004/024761 A1 which is directed to HAS-polypeptide-conjugates comprising one or more HAS molecules, wherein each HAS is conjugated to the polypeptide via a carbohydrate moiety or via a thioether. As possible linker to be coupled to a thiol group WO 2004024761 A1 mentions succinimidyl-(4-vinylsulfone)benzoate (SVSB), a linker comprising one vinylsulfon groups as well as an activated carboxylic acid group, namely an N-succinimidylester. In the method taught in WO 2004/024761 A1 a high excess of linker compound over HAS is employed (see e.g. Example 3, 2.3 Example Protocol 3 of WO 2004/024761).

However, the presence of multiple, potentially reactive, hydroxyl groups in the activated carboxylic acid derivative of the hydroxyalkyl starch can result in intra- or intermolecular bond formation between hydroxyalkyl starch molecules, e.g. between the oxidized reducing end and hydroxyl groups, so potentially undesired by-products may be formed, which are sometimes hard or even impossible to be purified away from the desired product (see working example E13 below). This is particularly true if the linker is employed in a molar excess when being coupled to HAS. If a linker is coupled to hydroxyalkyl starch using activated carboxylic acid chemistry, a product mixture may potentially be obtained by reactions with the multiple hydroxyl groups. This mixture may contain macromolecules carrying different numbers of linker molecules and/or variations in their attachment position within the macromolecules. So often a lower yield for the desired conjugation reaction between HAS and the conjugation partner results and, in particular, an inhomogeneous composition potentially comprising crosslinked polymer side products is obtained. In addition, in these cases, the described oxidation of the reducing end of hydroxyalkyl starch is considered to be not completely selective, thus also for this reason, potentially inhomogeneous products may result. Further, in some of the ligation methods taught in the art, the resulting conjugates are disadvantageous as regards the efficiency of the method and/or in particular as regards the stability of the resulting derivatives. Further, some linking strategies taught in the art, e.g. the maleimide-thio-linkage, are considered to have unpleasant side effects such as (unwanted) immunogenicity, a low stability of the thiol-reactive functional group during storage and/or under reductive conditions (such as disulfide bonds) and/or in the conjugation reaction and the like.

Thus, there is still the need for advantageous hydroxyalkyl starch derivatives for coupling to thiol groups of further compounds, which can be formed in a highly selective manner, which highly selectively react with the further compound and/or with which a stable and biocompatible linkage to the further compound can be provided. Further, there is the need for a selective method for the preparation of such derivatives, in which possible side reactions such as inter- and intramolecular crosslinking are significantly diminished or avoided and with which the derivatives can be provided in a high yield and high purity.

It was thus an object of the present invention to provide hydroxyalkyl starch derivatives for coupling to thiol groups of further compounds which derivatives overcome the problems of the prior art as well as a method for preparing the same, in particular which method provides the desired derivatives in high yield and with high specificity and purity. It is a further object of the present invention to provide novel, stable and biocompatible hydroxyalkyl starch derivatives comprising a protein attached to HAS via a thiol group of the protein as well as a method for preparing the same, in particular which method provides the desired derivatives in high yield and with high specificity and purity.

Therefore, the present invention relates to a hydroxyalkyl starch (HAS) derivative of formula (I)

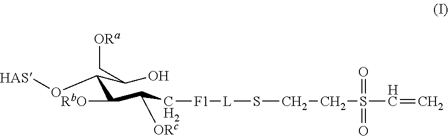

wherein
F1 is a functional group comprising the group —NR′—, with R′ being H or alkyl;
L is a spacer bridging F1 and S;
HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6.

It is to be understood that if R^{a} is HAS″, the hydroxyalkyl starch molecule has a branching site at the C6 position of the reducing end.

Further, the present invention relates to a hydroxyalkyl starch (HAS) derivative of formula (IV)

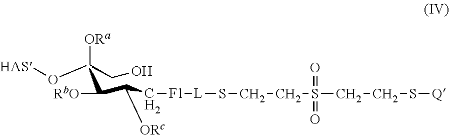

wherein
-Q′ is the remainder of a thiol group comprising compound Q which is linked via the group —S— of the thiol group to the —CH₂— group;
F1 is a functional group comprising the group —NR′—, with R′ being H or alkyl;
L is a spacer bridging F1 and S;
HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6.

The present invention also relates to a method for the preparation of a hydroxyalkyl starch (HAS) derivative, and a hydroxyalkyl starch (HAS) derivative obtained or obtainable by said method, said method comprising
(i) reacting hydroxyalkyl starch (HAS) of formula (Ia)

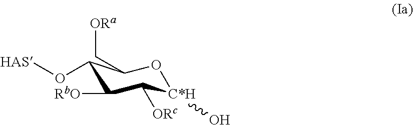

via carbon atom C* of the reducing end of the HAS with the functional group M of a crosslinking compound according to formula (II)

M-L-S-T (II)
wherein
M comprises the group —NHR′, with R′ being H or alkyl;
L is a spacer bridging M and S;
T is H or a thiol protecting group PG;
HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6,
thereby obtaining a HAS derivative according to formula (Ib)

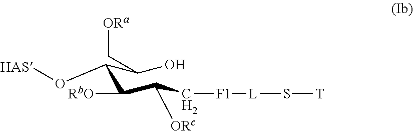

wherein —CH₂—F1- is the moiety resulting from the reaction of the group M with the HAS via the carbon atom C* of the reducing end, and F1 is a functional group comprising the group —NR′—; optionally removing PG in case T is PG to give T=H;
(ii) reacting the HAS derivative of formula (Ib) with a crosslinking compound of formula (III)

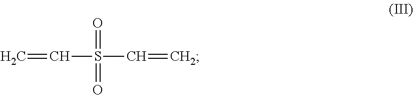

thereby obtaining a HAS derivative of formula (I)

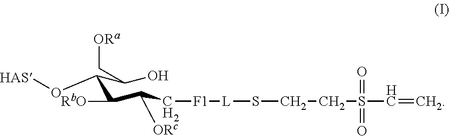

It has surprisingly been found that the derivatives according to formula (I) in which HAS is linked via its non-oxidized reducing end via a linker comprising an —NR′— group and a group —S—, said group —S— being linked to a specific sulfon group comprising linking moiety derived from divinyl sulfone were surprisingly stable while at the same time being selective and surprisingly reactive towards thiol groups of further compounds. Further, when compared to other linker compounds comprising vinylsulfone groups, divinyl sulfone of formula (III) showed surprisingly a superior chemoselectivity combined with a high degree of derivatization, thus reacted highly selective with the SH group of the HAS derivative of formula (Ib) yielding a highly pure product.

Further, the reaction of hydroxyalkyl starch (HAS) of formula (Ia) with the crosslinking compound according to formula (II) showed an surprising selectivity for the reducing end of HAS.

In particular, the resulting derivatives according to formula (IV) prepared using the derivatives of formula (I) were obtained with surprisingly high yields and/or high purity and/or showed a surprisingly high stability (see e.g. FIGS. 4-6) over a broad pH range, in particular at a physiological pH or lower.

Further, derivatives according to formula (IV) (which may hereinunder also be referred to as “conjugates”) comprising a protein as compound Q surprisingly showed essentially the same activity in biological assays than the protein as such (see e.g. examples C2 to C4 hereinunder). Thus, these conjugates are highly advantageous since it is contemplated that the hydroxyalkyl starch prolongs the circulation time of the active agent in the body.

### Hydroxyalkyl Starch

Hydroxyalkyl starch is an ether derivative of optionally partially hydrolyzed native starches wherein hydroxyl groups of the starch are suitably hydroxyalkylated. As hydroxyalkyl starches, hydroxypropyl starch and hydroxyethyl starch are preferred, with hydroxyethyl starch being most preferred.

Starch is a well-known polysaccharide according to formula (C₆H₁₀O₅)ₙ which essentially consists of alpha-D glucose units which are coupled via glycosidic linkages. Usually, starch essentially consists of amylose and amylopectin. Amylose consists of linear chains wherein the glucose units are linked via alpha-1,4-glycosidic linkages. Amylopectin is a highly branched structure with alpha-1,4-glycosidic linkages and alpha-1,6-glycosidic linkages.

Native starches from which hydroxyalkyl starches can be prepared include, but are not limited to, cereal starches and potato starches. Cereal starches include, but are not limited to, rice starches, wheat starches such as einkorn starches, spelt starches, soft wheat starches, emmer starches, durum wheat starches, or kamut starches, corn starches, rye starches, oat starches, barley starches, triticale starches, spelt starches, and millet starches such as sorghum starches or teff starches. Preferred native starches from which hydroxyalkyl starches are prepared have a high content of amylopectin relative to amylose. The amylopectin content of these starches is, for example, at least 70% by weight, preferably at least 75% by weight, more preferably at least 80% by weight, more preferably at least 85% by weight, more preferably at least 90% by weight such as up to 95% by weight, up to 96% by weight, up to 97% by weight, up to 98% by weight, up to 99% by weight, or up to 100% by weight. Native starches having an especially high amylopectin content are, for example, suitable potato starches such as waxy potato starches which are preferably extracted from essentially amylose-free potatoes which are either traditionally bred (e.g. the natural variety Eliane) or genetically modified amylopectin potato varieties, and starches of waxy varieties of cereals such as waxy corn or waxy rice.

A preferred hydroxyalkyl starch of the present invention has a constitution according to formula (Ia)

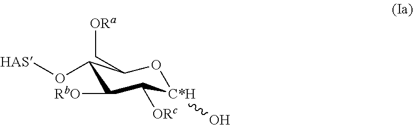

wherein HAS′ is the remainder of the HAS molecule and R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6.

According to a preferred embodiment, R¹, R², R³, and R⁴ are, independently of each other, selected from the group consisting of hydrogen and a methyl group, more preferably all of R¹, R², R³, and R⁴ are H.

Integer m is of from 2 to 4, such as 2, 3 or 4, preferably m is 2.

Integer n is of from 0 to 20, preferably of from 0 to 4, more preferably 0, 1, 2 or 3, most preferably 0.

According to a particularly preferred embodiment of the invention, the HAS derivative is a hydroxyethyl starch (HES) derivative. In this case, R¹ and R² are hydrogen, m is 2, n is 0 to 6, namely 0, 1, 2, 3, 4, 5, or 6, and R^{a}, R^{b}, R^{c} are the same or different from each other. Preferably, R^{b} and R^{c} are —[(CR¹R²)ₘO]ₙ—H and R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule, with n being 0 to 6, namely 0, 1, 2, 3, 4, 5 or 6, wherein in each group R^{a}, R^{b}, R^{c}, and n are the same or different from each other.

In formula (Ia) the reducing end of the starch molecule is shown in the non-oxidized form and the terminal saccharide unit of HAS is shown in the hemiacetal form which depending on e.g. the solvent, may be in equilibrium with the (free) aldehyde form. The abbreviation HAS′ as used in the context of the present invention refers to the HAS molecule without the terminal saccharide unit at the reducing end of the HAS molecule. This is meant by the term “remainder of the hydroxyalkyl starch molecule” as used herein.

The term “hydroxyalkyl starch” within the meaning of the present invention is not limited to compounds where the terminal carbohydrate moiety comprises groups R^{a}, R^{b} and/or R^{c} being —[(CR¹R²)ₘO]ₙ—H and/or HAS″ as depicted, for the sake of brevity, in formula (Ia), but refers to compounds in which at least one hydroxy group which is present anywhere else in the hydroxyalkyl starch, i.e. either in the terminal saccharide unit of the hydroxyalkyl starch molecule and/or in the remainder of the hydroxyalkyl starch molecule, HAS′, is substituted by a group —[(CR¹R²)ₘO]ₙ—H.

It is to be understood that the integer m in each group —[(CR¹R²)ₘO]ₙ—H present in the HAS molecule may be the same or may be different. The same applies to integer n.

The HAS may further contain one or more hydroxyalkyl groups, which comprise more than one hydroxyl group, in particular two or more hydroxyl groups. According to a preferred embodiment, the hydroxyalkyl groups comprised in HAS contain one hydroxy group only.

According to a preferred embodiment of the present invention, hydroxyalkyl starch according to above-mentioned formula (Ia) is employed. The other saccharide ring structures comprised in HAS′ may be the same as or different from the explicitly described saccharide ring, with the difference that they lack a reducing end.

HAS, in particular HES, is mainly characterized by the molecular weight distribution, the degree of substitution and the ratio of C₂/C₆ substitution.

There are two possibilities of describing the substitution degree. The degree of substitution (DS) of HAS, preferably HES, is described relatively to the portion of substituted glucose monomers with respect to all glucose moieties.

The substitution pattern of HAS, preferably HES, can also be described as the molar substitution (MS), wherein the number of hydroxyethyl groups per glucose moiety is counted.

In the context of the present invention, the substitution pattern of HAS, preferably HES, is referred to as MS, as described above (see also Sommermeyer et al., 1987, Krankenhauspharmazie, 8(8), 271-278, in particular p. 273).

MS is determined by gas chromatography after total hydrolysis of the HAS molecule, preferably the HES molecule. MS values of the respective HAS starting material, in particular the HES starting material, are given. It is assumed that the MS value is not affected during the method according to the invention.

HAS and in particular HES solutions are present as polydisperse compositions, wherein each molecule differs from the other with respect to the polymerization degree, the number and pattern of branching sites, and the substitution pattern. HAS and in particular HES is therefore a mixture of compounds with different molecular weight. Consequently, a particular HAS solution, and preferably a particular HES solution, is determined by the average molecular weight with the help of statistical means. In this context, Mₙ is calculated as the arithmetic mean depending on the number of molecules and their molecular weight. Alternatively, the mass distribution in HAS, and in particular HES, may be described by the weight average molecular weight M_{w} (or Mw).

### The Parameter Mₙ

The number average molecular weight is defined by the following equation:

Mₙ=ΣᵢnᵢMᵢ/Σᵢnᵢ

wherein nᵢ is the number of hydroxyalkyl starch molecules of species i having molar mass Mᵢ.

### The Parameter M_{w}

The weight average molecular weight is defined by the following equation:

M_{w}=ΣᵢnᵢMᵢ²/ΣᵢnᵢMᵢ

wherein nᵢ is the number of hydroxyalkyl starch molecules of species i having molar mass Mᵢ. According to the present invention, typical M_{w} values are preferably in the range of from 1 to 1000 kDa, more preferably of from 1 to 800 kDa, more preferably of from 1 to 700 kDa, more preferably of from 2 to 600 kDa, more preferably of from 5 to 500 kDa, most preferably of from 25 to 400 kDa.

### The Parameter MS

The second parameter which is usually referred to as “MS” (molecular substitution) describes the number of hydroxyalkylated sites per anhydroglucose unit of a given hydroxyalkyl starch (Sommermeyer et al., Krankenhauspharmazie 8 (8), 1987, pp 271-278, in particular page 273). The values of MS correspond to the degradability of the hydroxyalkyl starch by alpha-amylase. Generally, the higher the MS value of the hydroxyalkyl starch, the lower is its respective degradability.

The parameter MS can be determined according to Ying-Che Lee et al., Anal. Chem. 55, 1983, pp 334-338; or K. L. Hodges et al., Anal. Chem 51, 1979, p 2171. According to these methods, a known amount of the hydroxyalkyl starch is subjected to ether cleavage in xylene whereby adipinic acid and hydriodic acid are added. The amount of released iodoalkane is subsequently determined via gaschromatography using toluene as an internal standard and iodoalkane calibration solutions as external standards. According to the present invention, typical MS values are in the range of from 0.1 to 3, preferably of from 0.4 to 1.3, such as 0.4, 0.5, 0.6, 0.7, 0.8, 0.9, 1.0, 1.1, 1.2 or 1.3.

### The Parameter C2/C6 Ratio

The third parameter which is referred to as “C2/C6 ratio” describes the ratio of the number of the anhydroglucose units being substituted in C2 position relative to the number of the anhydroglucose units being substituted in C6 position. During the preparation of the hydroxyalkyl starch, the C2/C6 ratio can be influenced via the pH used for the hydroxyalkylation reaction. Generally, the higher the pH, the more hydroxyl groups in C6 position are hydroxyalkylated. The parameter C2/C6 ratio can be determined, for example, according to Sommermeyer et al., Krankenhauspharmazie 8 (8), 1987, pp 271-278, in particular page 273.

According to the present invention, typical values of the C2/C6 ratio are in the range of from 2 to 20, preferably of from 2 to 15, more preferably of from 2 to 12.

### The Reducing End

According to a preferred embodiment the compound according to formula (II) is selectively reacted via carbon atom C* of the reducing end, i.e. with the reducing end of HAS. The term “selectively reacted with the reducing end” relates to processes according to which preferably at least 95%, more preferably at least 98%, more preferably at least 99%, more preferably at least 99.5%, more preferably at least 99.9% of all reacted HAS molecules are exclusively reacted via their reducing end group.

According to the present invention HAS is reacted via its non-oxidized reducing end.

### Step (i)

In step (i) of the method according to the invention, the HAS according to formula (Ia) is reacted via carbon atom C* of the reducing end of the HAS with the functional group M of a crosslinking compound according to formula M-L-S-T (II), wherein a HAS derivative according to formula (Ib) is obtained, wherein —CH₂—F1- is the moiety resulting from the reaction of the group M with the HAS via the carbon atom C* of the reducing end, and wherein F1 is a functional group comprising the group —NR′—.

The functional group F1 and the functional group M

M is a functional group comprising the moiety —NHR′, with R′ being H or alkyl. Preferably R′ is selected from the group consisting of H, methyl, ethyl and propyl.

According to a preferred embodiment of the invention, the functional group M of the crosslinking compound according to formula (II) is selected from the group consisting of CH₃—NH—, CH₃—CH₂—NH—, CH₃—CH₂—CH₂—NH—, (CH₃)₂—CH—NH—, H₂N—, H₂N—O—, H₂N—NH—, H₂N—NH—(C=G)-, H₂N—NH—(C=G)-G^{G}- and H₂N—NH—SO₂— with G being O, S or NR^{m} with R^{m} being H or alkyl, preferably with G being O; and with G^{G} being O, S or NR^{G} and with R^{G} being H or alkyl, in particular with G^{G} being O or NR^{G} with R^{G} being H, the crosslinking compound thus preferably having one of the following structures: CH₃—NH-L-S-T, CH₃—CH₂—NH-L-S-T, CH₃—CH₂—CH₂—NH-L-S-T, (CH₃)₂—CH—NH-L-S-T, H₂N-L-S-T, H₂N—O-L-S-T, H₂N—NH-L-S-T, H₂N—NH—(C=G)-L-S-T, H₂N—NH—(C=G)-G^{G}-L-S-T or H₂N—NH—SO₂-L-S-T, and more preferably one of the following structures: CH₃—NH-L-S-T, CH₃—CH₂—NH-L-S-T, CH₃—CH₂—CH₂—NH-L-S-T, (CH₃)₂—CH—NH-L-S-T, H₂N-L-S-T, H₂N—O-L-S-T, H₂N—NH-L-S-T, H₂N—NH—(C═O)-L-S-T, H₂N—NH—(C═O)—NH-L-S-T, H₂N—NH—(C═O)—O-L-S-T or H₂N—NH—SO₂-L-S-T.

Thus, the present invention also relates to a method for the preparation of a hydroxyalkyl starch derivative, as described above, wherein in step (i), the hydroxyalkyl starch (HAS) of formula (Ia) is reacted via carbon atom C* of the reducing end of the HAS with the functional group M of a crosslinking compound according to formula (II)

M-L-S-T (II)

wherein M of the crosslinking compound according to formula (II) is selected from the group consisting of CH₃—NH—, CH₃—CH₂—NH—, CH₃—CH₂—CH₂—NH—, (CH₃)₂—CH—NH—, H₂N—, H₂N—O—, H₂N—NH—, H₂N—NH—(C=G)-, H₂N—NH—(C=G)-G^{G}- and H₂N—NH—SO₂— with G being O, S or NR^{m} with R^{m} being H or alkyl, preferably with G being O; and with G^{G} being O, S or NR^{G} with R^{G} being H or alkyl, in particular with G^{G} being O or NR^{G} and with R^{G} being H, the crosslinking compound thus preferably having one of the following structures: CH₃—NH-L-S-T, CH₃—CH₂—NH-L-S-T, CH₃—CH₂—CH₂—NH-L-S-T, (CH₃)₂—CH—NH-L-S-T, H₂N-L-S-T, H₂N—O-L-S-T, H₂N—NH-L-S-T, H₂N—NH—(C=G)-L-S-T, H₂N—NH—(C=G)-G^{G}-L-S-T or H₂N—NH—SO₂-L-S-T, more preferably one of the following structures: CH₃—NH-L-S-T, CH₃—CH₂—NH-L-S-T, CH₃—CH₂—CH₂—NH-L-S-T, (CH₃)₂—CH—NH-L-S-T, H₂N-L-S-T, H₂N—O-L-S-T, H₂N—NH-L-S-T, H₂N—NH—(C═O)-L-S-T, H₂N—NH—(C═O)—NH-L-S-T, H₂N—NH—(C═O)—O-L-S-T or H₂N—NH—SO₂-L-S-T.

Thereby, a HAS derivative according to formula (Ib)

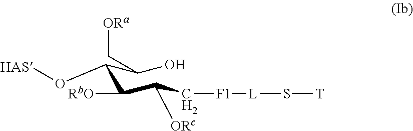

is obtained, wherein —CH₂—F1- is the moiety resulting from the reaction of the group M with the HAS via the carbon atom C* of the reducing end, and F1 is a functional group comprising the group —NR′—, with R′ being H or alkyl. Preferably F1 is —(CH₃)—N—, —(CH₃—CH₂)—N—, —(CH₃—CH₂—CH₂)—N—, —((CH₃)₂—CH)—N—, —HN—, —HN—O—, —HN—NH—, —HN—NH—(C=G)-, —HN—NH—(C=G)-G^{G}- and —HN—NH—SO₂—.

More preferably R′ is H, thus the functional group M of the crosslinking compound according to formula (II) is preferably a functional group comprising the moiety H₂N—, more preferably M is selected from the group consisting of H₂N—, H₂N—O—, H₂N—NH—, H₂N—NH—(C=G)-, H₂N—NH—(C=G)-G^{G}- and H₂N—NH—SO₂— with G being O, S or NR^{m} with R^{m} being H or alkyl, preferably with G being O; and with G^{G} being O, S or NR^{G} with R^{G} being H or alkyl, in particular with G^{G} being O or NR^{G} and with R^{G} being H, more preferably M is selected from the group consisting of H₂N—, H₂N—O—, H₂N—NH—, H₂N—NH—(C═O)—, H₂N—NH—(C═O)—NH—, H₂N—NH—(C═O)—O— and H₂N—NH—SO₂—; more preferably M is selected from the group consisting of H₂N—, H₂N—O—, H₂N—NH— and H₂N—NH—C(═O)—, more preferably M is H₂N—, the crosslinking compound thus having the structure H₂N-L-S-T.

Consequently, F1 is preferably selected from the group consisting of —HN—, —HN—O—, —HN—NH—, —HN—NH—(C=G)-, —HN—NH—(C=G)-G^{G}- and —HN—NH—SO₂— with G being O, S or NR^{m} with R^{m} being H or alkyl, preferably with G being O; and with G^{G} being O, S or NR^{G} with R^{G} being H or alkyl, in particular with G^{G} being O or NR^{G} and with R^{G} being H, more preferably F1 is selected from the group consisting of —HN—, —HN—O—, —HN—NH—, —HN—NH—(C═O)—, —HN—NH—(C═O)—NH—, —HN—NH—(C═O)—O— and —HN—NH—SO₂—; more preferably F1 is selected from the group consisting of —HN—, —HN—O—, —HN—NH— and —HN—NH—C(═O)—, more preferably F1 is —HN—. Thus, the present invention also relates to a HAS derivative, as described above, or a HAS derivative obtained or obtainable by a method as described above, wherein F1 is selected from the group consisting of —HN—, —HN—O—, —HN—NH—, —HN—NH—(C=G)-, —HN—NH—(C=G)-G^{G}- and —HN—NH—SO₂—, more preferably F1 is —HN—.

Thus, the present invention relates to a hydroxyalkyl starch (HAS) derivative, as described above, or a HAS derivative obtained or obtainable by the method as described above, wherein F1 is —NH—, the derivative thus having a structure according to the following formula:

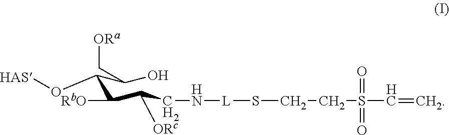

Further, the present invention relates to a hydroxyalkyl starch (HAS) derivative, as described above, or a HAS derivative obtained or obtainable by the method as described above, wherein F1 is —NH—, and wherein the derivative has a structure according to the following formula:

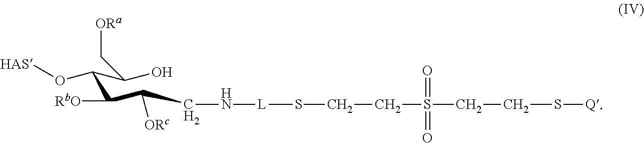

### The Spacer L

As described above L is a spacer bridging M and S or bridging F1 and S, respectively. Thus, in case the HAS derivatives described above are prepared by a method comprising the step (i), as described above, thus by reacting the reducing end of HAS with the crosslinking compound according to formula (II), L is first linking M and S in the crosslinking compound, and, subsequently, after reaction of the crosslinking compound of formula (II) with the reducing end, whereupon F1 is formed, L is linking the thus obtained functional group F1 and S.

Preferably, L comprises, more preferably consists of, an alkyl, alkenyl, alkylaryl, arylalkyl, aryl or heteroaryl group.

Within the meaning of the present invention, the term “alkyl” relates to non-branched alkyl residues, branched alkyl residues, cycloalkyl residues, as well as residues comprising one or more heteroatoms or functional groups, such as, by way of example, —O—, —S—, —NR^{L1}—, —NR^{L1}—C(═O)—, —C(═O)—NR^{L1}— and the like, with R^{L1} being alkyl, preferably methyl. The term also encompasses alkyl groups which are further substituted by one or more suitable substituent. The term “substituted alkyl” as used in this context of the present invention preferably refers to alkyl groups being substituted in any position by one or more substituents, preferably by 1, 2, 3, 4, 5 or 6 substituents, more preferably by 1, 2, or 3 substituents. If two or more substituents are present, each substituent may be the same as or different from the at least one other substituent. There are in general no limitations as to the substituent.

“Suitable substituents” in the context of spacer L are, for example, selected from the group consisting of alkyl, aryl, alkenyl, alkynyl, fluorine, hydroxyl, alkylcarbonyloxy, arylcarbonyloxy, alkoxycarbonyloxy, aryloxycarbonyloxy, carboxyl, alkoxycarbonyl, aminocarbonyl, alkylaminocarbonyl, dialkylaminocarbonyl, alkoxy, phosphate, phosphonato, phosphinato, tertiary amino, acylamino, including alkylcarbonylamino, arylcarbonylamino, carbamoyl, ureido, nitro, alkylthio, arylthio, amide, sulfate, alkylsulfinyl, sulfonate, sulfonamido, trifluoromethyl, cyano, azido, carboxymethylcarbamoyl [i.e. the group —C(═O)(—NH—CH₂—COOH)], cycloalkyl such as e.g. cyclopentyl or cyclohexyl, heterocycloalkyl such as e.g. morpholino, piperazinyl or piperidinyl, alkylaryl, arylalkyl and heteroaryl. Preferred substituents of such organic residues are, for example, alkyl groups, amide groups, hydroxyl groups, and carboxyl groups.

It is to be understood that, within the meaning of the present invention, the term suitable substituents as used hereinunder and above also includes suitable salts of the respective substituents.

The term “alkenyl” as used in the context of the present invention refers to unsaturated alkyl groups having at least one double bond. The term also encompasses alkenyl groups which are substituted by one or more suitable substituent.

The term “alkynyl” refers to unsaturated alkyl groups having at least one triple bond. The term also encompasses alkynyl groups which are substituted by one or more suitable substituent.

Within the meaning of the present invention, the term “aryl” refers to, but is not limited to, optionally suitably substituted 5- and 6-membered single-ring aromatic groups as well as optionally suitably substituted multicyclic groups, for example bicyclic or tricyclic aryl groups. The term “aryl” thus includes, for example, optionally suitably substituted phenyl groups or optionally suitably substituted naphthyl groups. Aryl groups can also be fused or bridged with alicyclic or heterocycloalkyl rings which are not aromatic so as to form a polycycle, e.g., benzodioxolyl or tetraline.

The term “heteroaryl” as used within the meaning of the present invention includes optionally suitably substituted 5- and 6-membered single-ring aromatic groups as well as substituted or unsubstituted multicyclic aryl groups, for example bicyclic or tricyclic aryl groups, comprising one or more, preferably from 1 to 4, such as 1, 2, 3 or 4, heteroatoms, wherein in case the aryl residue comprises more than 1 heteroatom, the heteroatoms may be the same or different. Such heteroaryl groups including from 1 to 4 heteroatoms are, for example, benzodioxolyl, pyrrolyl, furanyl, thiophenyl, thiazolyl, isothiazolyl, imidazolyl, triazolyl, tetrazolyl, pyrazolyl, oxazolyl, isoxazolyl, pyridinyl, pyrazinyl, pyridazinyl, benzoxazolyl, benzodioxazolyl, benzothiazolyl, benzoimidazolyl, benzothiophenyl, methylenedioxyphenyl, napthyridinyl, quinolinyl, isoquinolinyl, indolyl, benzofuranyl, purinyl, benzofuranyl, deazapurinyl, or indolizinyl.

The terms “substituted aryl” and “substituted heteroaryl” as used in the context of the present invention describe moieties having suitable substituents replacing a hydrogen atom on one or more atoms, e.g. C or N, of an aryl or heteroaryl moiety.

Preferably, the spacer L comprises the moiety —(C(L′L″))_{q}- with L′ and L″ in each repeating unit —C(L′L″)- being, independently of each other, selected from the group consisting of H, alkyl, aryl, alkenyl, alkynyl, hydroxyl, fluorine, alkylcarbonyloxy, arylcarbonyloxy, alkoxycarbonyloxy, aryloxycarbonyloxy, amide, carboxyl, alkoxycarbonyl, aminocarbonyl, alkylaminocarbonyl, dialkylaminocarbonyl, alkoxy, phosphate, phosphonato, phosphinato, tertiary amino, acylamino, including alkylcarbonylamino, arylcarbonylamino, carbamoyl, ureido, nitro, alkylthio, arylthio, sulfate, alkylsulfinyl, sulfonate, sulfonamido, trifluoromethyl, cyano, azido, carboxymethylcarbamoyl [i.e. the group —C(═O)(—NH—CH₂—COOH)], cycloalkyl such as e.g. cyclopentyl or cyclohexyl, heterocycloalkyl such as e.g. morpholino, piperazinyl or piperidinyl, alkylaryl, arylalkyl and heteroaryl, wherein the groups L′ and L″ in each repeating unit may be the same or may differ from each other, with q preferably being in the range of from 1 to 20, more preferably in the range of from 1 to 10, more preferably in the range of from 2 to 6, more preferably, 2, 3 or 4.

According to a preferred embodiment of the invention, L′ and L″ are, independently of each other, selected from the group consisting of H, alkyl groups (including substituted alkyl groups, in particular including hydroxyalkyl groups), amide groups, hydroxyl groups, and carboxyl groups, wherein the groups L′ and L″ in each repeating unit may be the same or may differ from each other.

According to a particularly preferred embodiment of the invention, L′ and L″ are, independently of each other, selected from the group consisting of H, amide, carboxyl and alkyl (including substituted alkyl groups, in particular including hydroxyalkyl groups), more preferably L′ and L″ are H or alkyl, such as H or methyl, wherein the groups L′ and L″ in each repeating unit may be the same or may differ from each other.

More preferably, L has a structure selected from the group consisting of

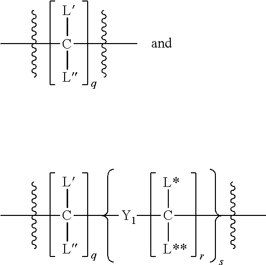

with q preferably being in the range of from 1 to 20, wherein L′, L″, L* and L**, are independently of each other, selected from the group consisting of H, alkyl, aryl, alkenyl, alkynyl, hydroxyl, fluorine, alkylcarbonyloxy, arylcarbonyloxy, alkoxycarbonyloxy, aryloxycarbonyloxy, amide, carboxyl, alkoxycarbonyl, aminocarbonyl, alkylaminocarbonyl, dialkylaminocarbonyl, alkoxy, phosphate, phosphonato, phosphinato, tertiary amino, acylamino, including alkylcarbonylamino, arylcarbonylamino, carbamoyl, ureido, nitro, alkylthio, arylthio, sulfate, alkylsulfinyl, sulfonate, sulfonamido, trifluoromethyl, cyano, azido, carboxymethylcarbamoyl [i.e. the group —C(═O)(—NH—CH₂—COOH)], cycloalkyl such as e.g. cyclopentyl or cyclohexyl, heterocycloalkyl such as e.g. morpholino, piperazinyl or piperidinyl, alkylaryl, arylalkyl and heteroaryl, wherein the groups L′ and L″ in each repeating unit may be the same or may differ from each other, with q preferably being in the range of from 1 to 20, more preferably in the range of from 1 to 10, more preferably in the range of from 2 to 6, more preferably, 2, 3 or 4, and with r preferably being in the range of from 1 to 10 and with s preferably being in the range of from 1 to 10, and wherein Y₁ is a functional moiety selected from the group consisting of —O—, —S—, —NR^{Y1}—, —NH—C(═O)—, —C(═O)—NH—,

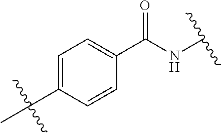 [SMILES: *NC(=O)c1ccc(*)cc1]

(“p-phenyl-C(═O)—NH”),
and wherein R^{Y1} is alkyl, preferably methyl, and wherein the groups L′, L″, L* and L**, in each repeating unit, may be the same or may differ from each other.

L may have one or more asymmetric centers. As consequence, the linker may be employed as mixtures of enantiomers and as individual enantiomers, as well as diastereomers and mixtures of diastereomers. All possible stereoisomers, single isomers and mixtures of isomers are included within the scope of the present invention. In case L comprises one or more asymmetric centers, L is preferably employed in enantiomeric or diastereomeric pure form.

Preferably, L′, L″, L* and L** are, independently of each other, selected from the group consisting of H, alkyl groups (including substituted alkyl groups, in particular including hydroxyalkyl groups), amide groups, hydroxyl groups, and carboxyl groups, more preferably from H, amide including —C(═O)—NH₂, carboxyl, hydroxyl and alkyl, in particular L′, L″, L* and L** are, independently of each other, H or alkyl, and wherein Y₁ is a functional moiety as described above, preferably wherein Y is O, —NH—C(═O)— or —C(═O)—NH—. In case q is >1, the repeating units —(C(L′L″))- may be the same or may be different from each other. In case r is >1, the repeating units —(C(L*L**))- may be the same or may be different from each other. In case s is >1, the repeating units —Y₁—[C(L*L**)]ᵣ- may be the same or may be different from each other.

According to one preferred embodiment, L has the structure

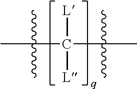

wherein L′ and L″ are, independently of each other, selected from H and alkyl, wherein in each repeating unit (C(L′L″))-, in case q is >1, L′ and L″ may be the same or may be different from each other.

As example, without being meant to be limiting, the following groups L are mentioned: —CH₂—, —CH₂—CH₂—, —CH₂—CH₂—CH₂—, —CH₂—CH₂—CH₂—CH₂—, —CH₂—CH₂—CH₂—CH₂—CH₂—, —CH₂—CH₂—CH₂—CH₂—CH₂—CH₂—, —CH₂—CH₂—CH₂—CH₂—CH₂—CH₂—CH₂—, —CH₂—CH₂—CH₂—CH₂—CH₂—CH₂—CH₂—CH₂—, —CH₂—CH₂—CH₂—CH₂—CH₂—CH₂—CH₂—CH₂—CH₂—, —CH₂—CH₂—CH₂—CH₂—CH₂—CH₂—CH₂—CH₂—CH₂—CH₂—, —CH(CH₃)—, —CH(CH₃)—CH₂—, —CH₂—CH(CH₃)—, —CH(CH₃)—CH₂—CH₂—, —CH₂—CH(CH₃)—CH₂—, —CH₂—CH₂—CH(CH₃)—, —CH(CH₃)—CH₂—CH₂—CH₂—, —CH₂—CH(CH₃)—CH₂—CH₂—, —CH₂—CH₂—CH(CH₃)—CH₂—, —CH₂—CH₂—CH₂—CH(CH₃)—, —CH(CH₃)—CH₂—CH₂—CH₂—CH₂—, —CH₂—CH(CH₃)—CH₂—CH₂—CH₂—, —CH₂—CH₂—CH(CH₃)—CH₂—CH₂—, —CH₂—CH₂—CH₂—CH(CH₃)—CH₂—, —CH₂—CH₂—CH₂—CH₂—CH(CH₃)—, —CH(CH₃)—CH₂—CH₂—CH₂—CH₂—CH₂—, —CH₂—CH(CH₃)—CH₂—CH₂—CH₂—CH₂—, —CH₂—CH₂—CH(CH₃)—CH₂—CH₂—CH₂—, —CH₂—CH₂—CH₂—CH(CH₃)—CH₂—CH₂—, —CH₂—CH₂—CH₂—CH₂—CH(CH₃)—CH₂—, —CH₂—CH₂—CH₂—CH₂—CH₂—CH(CH₃)—, —CH₂—C(CH₃)₂—CH₂—, —CH(CH₃)—CH(CH₃)—, —C(CH₃)₂—C(CH₃)₂—, —CH(CH₂OH)—CH₂—, —CH(CH₂OH)—CH₂—CH₂—, —CH(CONH₂)—CH₂—, —CH(COOH)—CH₂—, —CH(COOH)—CH₂—CH₂—, —CH(COOH)—CH₂—CH₂—CH₂—CH₂—, —CH(CONH₂)—C(CH₃)₂—, —CH(CONH₂)—CH₂—CH₂—CH₂—CH₂—, —CH₂—CH(OH)—CH₂—, —CH₂—CH(OH)—CH(OH)—CH₂— and —CH(COOH)—C(CH₃)₂—.

More preferably L′ and L″ are in each repeating unit H.

According to a particularly preferred embodiment of the invention, L is —CH₂—CH₂—.

According to an alternative embodiment, as described above, L has as structure according to the following formula

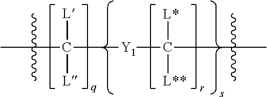

with q preferably being in the range of from 1 to 20, with r preferably being in the range of from 1 to 10, with s preferably being in the range of from 1 to 10 and with L′, L″, L* and L** being as described above.

As regards the functional moiety Y₁, said functional moiety is preferably selected from the group consisting of —O—, —S—, —NR^{Y1}—, —NH—C(═O)—, —C(═O)—NH—, p-phenyl-C(═O)—NH with R^{Y1} in particular being methyl, more preferably wherein Y₁ is —O—, —NH—C(═O)— or —C(═O)—NH—.

According to one preferred embodiment of the present invention, s is 1. Thus, L is, for example, —[C(L′L″)]_{q}-N(CH₃)—[C(L*L**)]ᵣ-, —[C(L′L″)]_{q}—S—[C(L*L**)]ᵣ, —[C(L′L″)]_{q}-N(CH₃)—[C(L*L**)]ᵣ-, —[C(L′L″)]_{q}-NH—C(═O)—[C(L*L**)]ᵣ-, —[C(L′L″)]_{q}—C(═O)—NH—[C(L*L**)]ᵣ- or —[C(L′L″)]_{q}-p-phenyl-C(═O)—NH—[C(L*L**)]ᵣ-. Most preferably q is in the range of from 2 to 4. Further, r is in particular in the range of from 2 to 4. According to a preferred embodiment, Y₁ is —O— or —C(═O)—NH—.

Thus, as example, without being meant to be limiting, the following groups L are mentioned: —CH₂—CH₂—O—CH₂—CH₂—, —CH₂—CH₂—O—CH₂—CH₂—O—CH₂—CH₂—, —CH₂—CH₂—O—CH₂—CH₂—O—CH₂—CH₂—O—CH₂—CH₂—, and —CH(COOH)—CH₂—CH₂—C(═O)NH—CH—C(═O)(—NH—CH₂—COOH)—CH₂—.

According to a further preferred embodiment, s is >1, preferably of from 2 to 10. In this case, Y₁ is preferably —O—, —C(═O)—NH— or —NH—C(═O)—. Thus, this embodiment includes any spacer L derived from a peptide, thus having a peptidic backbone. In case L is derived from a peptidic crosslinking compound (II), the crosslinking compound (II) is preferably employed in enantiomeric or diastereomeric pure form, more preferably in the natural occurring stereoisomeric form.

In step (i) HAS is preferably dissolved in an aqueous medium, more preferably in a reaction buffer, and a crosslinking compound according to formula (II) is subsequently added.

Preferably, the reaction in (i) is carried out in a solvent selected from the group consisting of DMSO, DMF, DMA, NMP, formamide, water, reaction buffers and mixtures of two or more thereof. Preferred reaction buffers are, e.g., sodium citrate buffer, sodium acetate buffer, sodium phosphate buffer, sodium carbonate buffer, or sodium borate buffer. Preferred pH values of the reaction buffers are in the range of from 4 to 9, more preferably of from 5 to 7. The pH values given hereinunder and above refer to pH values determined via a pH sensitive glass electrode.

The crosslinking compound of formula (II) is preferably used as free amine or as a salt and added as solid. Preferred are salts such as the hydrochloride, acetate or trifluoroacetate salt.

In step (i) preferably an excess of the crosslinking compound of formula (II) is employed. Preferably, the minimum amount of crosslinking compound (II) is one molar equivalent with respect to the amount of reducing ends to be reacted. The maximum amount is given by the solubility limit of the crosslinking compound in the particular reaction solvents. Preferably, the crosslinking compound is employed at a concentration in the range of at least 0.05 mol/l, preferably at least 0.1 mol/l, more preferably in the range of from 0.2 to 4 mol/l, more preferably from 0.5 to 2 mol/l, more preferably 0.9 mol/l to 1.1 mol/l, most preferably about 1 mol/1.

The reaction mixture is preferably stirred at a temperature in the range of from 5° C. to 100° C., more preferably at a temperature in the range of from 20° C. to 90° C., more preferably in the range of from 40° C. to 80° C. During the course of the reaction, the temperature may be varied, preferably in the above-given ranges, or held essentially constant.

The reaction is preferably conducted for a time in the range of from 1 to 48 h, more preferably from 2 to 36 h, more preferably from 4 to 18 h.

Upon reaction of M which comprises the NHR′— group with the reducing end, initially a functional group F1*is formed, said functional group comprising the structure —CR′═N—. For example, in case M consists of an NH₂— group, initially a functional group F1* is formed, said functional group having the structure —CH═N—. In case M is an NH₂—NH-group, the functional group F1* which is formed upon reaction of M with the reducing end, is a —CH═N—NH— group. In case M is an NH₂—O— group, the functional group F1* which is formed upon reaction of M with the reducing end, is a —CH═N—O— group.

The hydroxyalkyl starch derivative obtained, i.e. the hydroxyalkyl starch derivative comprising the group F1*, said group comprising the structure —CR′═N—, preferably being selected from the group consisting of —CH═N—NH—, —CH═N— and —CH═N—O—, may be isolated from the reaction mixture by ultrafiltration or dialysis, preferably ultrafiltration followed by lyophilization of the isolated hydroxyalkyl starch derivative.

In case this HAS derivative which comprises the functional group F* contains a free thiol group (i.e. T=H), the ultrafiltration or dialysis is preferably carried out under neutral conditions, preferably in water. Further, the hydroxyalkyl starch derivative may be precipitated from the reaction mixture, in particular by adding an alcohol, preferably 2-propanol. The obtained precipitate may be collected by filtration or centrifugation and may further be purified using conventional purification protocols, preferably ultrafiltration, dialysis or chromatographic methods, preferably size exclusion chromatography.

According to the invention, step (i) preferably additionally comprises the conversion of the, optionally isolated, hydroxyalkyl starch derivative obtained upon reaction of HAS with the crosslinking compound according to formula (II) prior to step (ii). In this step the functional group F1*obtained upon reaction of M with the reducing end is suitably reduced, to give the functional group F1. For example, in case the linking group is a —CH═N— group, said group is reduced to give the group F1 with F1 being —CH₂—NH—. In case, the linking group is a —CH═N—NH₂— group, said group is reduced to give the group F1 with F1 being —CH₂—NH—NH₂—. In case, the linking group is a —CH═N—O— group, said group is reduced to give the group F1 with F1 being —CH₂—NH—O—.

The reduction is preferably carried out in the presence of a suitable reducing agent, such as sodium borohydride, sodium cyanoborohydride, sodium triacetoxy borohydride, organic borane complex compounds such as a 4-(dimethylamino)pyridine borane complex, N-ethyldiisopropylamine borane complex, N-ethylmorpholine borane complex, N-methylmorpholine borane complex, N-phenylmorpholine borane complex, lutidine borane complex, triethylamine borane complex, or trimethylamine borane complex, preferably sodium cyanoborohydride.

Preferably, this reduction is carried out using an excess of reducing agent, so preferably a minimum of one molar equivalent with respect to the amount of reducing ends in HAS is applied. More preferably, the concentration of the reducing agent used for this reaction of the present invention is in the range of from 0.001 to 3.0 mol/l, more preferably in the range of from 0.05 to 2.0 mol/l, more preferably in the range of from 0.1 to 1 mol/l, more preferably in the range of 0.3-0.6 mol/L, relating, in each case, to the volume of the reaction solution.

The reduction, as described above, can either be carried out subsequently to the coupling process, in which M is coupled to the reducing end, optionally after isolating the coupled product prior to the reduction, or it is possible to carry out the same reaction all in one pot, with the coupling to the reducing end and the reduction occurring concurrently. Most preferably the above mentioned one pot synthesis is carried out. Both reactions are referred to in the context of the present invention as “reductive amination”.

Thus, the present invention also relates to a method as described above, and a conjugate obtained or obtainable by said method, wherein the functional group M comprises the group HR′N—, preferably H₂N—, more preferably consist of the group H₂N— and the reaction according to step (i) is a reductive amination. Further, the present invention also relates to a method as described above, and a conjugate obtained or obtainable by said method, wherein the functional group M is H₂N—NH— and the reaction according to step (i) is a reductive amination.

Since, the coupling and reduction, as described above, are preferably carried out in one pot, the solvent used for the reduction step is preferably also selected from the solvents already mentioned above in the context of step (i). Thus preferably, the reductive amination is carried out in a solvent selected from the group consisting of DMSO, DMF, DMA, NMP, formamide, water, acetic acid reaction buffers and mixtures of two or more thereof. Preferred pH values of are thus in the range of from 4 to 9, more preferably of from 5 to 8.

During the reductive amination reaction, the temperature of the reaction mixture is suitably chosen. Generally, during the reductive amination reaction, the temperature of the reaction mixture is in the range of from 5 to 100° C. such as from 20 to 90° C. or from 40 to 80° C. Preferably, during the reductive amination reaction, the temperature of the reaction mixture is in the range of from 45 to 75° C., more preferably from 55 to 65° C. The reductive amination reaction can be carried out for any suitable time period. Generally, the time period is in the range of from 1 to 48 h such as from 2 to 36 h. Preferably, the time period is in the range of from 3 to 24 h, more preferably from 6 to 21 h, more preferably from 4 to 18 h. Preferably, to reductive amination is carried out at a temperature of the reaction mixture in the range of from 40 to 90° C. for a time period of from 1 to 36 h, more preferably at a temperature in the range of from 45 to 80° C. for a time period of from 2 h to 24 h, more preferably at a temperature in the range of from 55 to 65° C. for a time period of from 4 to 18 h.

Thus, the present invention also relates to a method, as described above, and a HAS derivative obtained or obtainable by said method, wherein the reacting according to step (i) is carried out under reductive amination conditions, preferably at a temperature in the range of from 5° C. to 100° C. and in a solvent selected from the group consisting of DMSO, DMF, DMA, NMP, formamide, water, acetic acid, and reaction buffers and mixtures of two or more thereof.

According to above-described preferred embodiment wherein the reaction of the crosslinking compound (II) with HAS is carried out under reductive amination conditions, the concentration of HAS, preferably HES, in the aqueous system is preferably in the range of at least 1 weight-%, more preferably at least 10 weight-%, more preferably in the range of from 20-40 weight-%, most preferably around 30 weight-%, based on the total weight of the whole solution.

After having finished the reductive amination reaction, the reaction mixture obtained is preferably subjected to a suitable work up. Such working up may comprise one or more stages wherein preferably at least one stage comprises a purification, preferably a purification by ultrafiltration, precipitation, size exclusion chromatography, and a combination of two or more of these methods, more preferably by ultrafiltration. Optionally, such working up may comprise at least one stage which comprises a pH adjustment, preferably an adjustment to a pH of at least 8, preferably at least 9, more preferably in the range of from 9 to 11. Adjusting the pH of the reaction mixture to a value of at least 8, preferably at least 9, more preferably from 9 to 11 can be realized, if carried out, according to all conceivable methods. Preferably, an inorganic base, preferably an alkali metal base and/or an alkaline earth metal base, more preferably an alkali metal hydroxide and/or an alkaline earth metal hydroxide, more preferably an alkaline metal hydroxide, more preferably sodium hydroxide is added in a suitable amount. The addition of such a basic compound can be performed at the temperature of the reaction mixture of the reductive amination reaction. Preferably, the reaction mixture obtained from the reductive amination reaction is cooled before the basic compound is added, preferably to a temperature in the range of from 10 to 35° C., more preferably from 20 to 30° C. During adding the basic compound, the mixture can be suitably stirred. The pH is to be understood as the value indicated by a pH sensitive glass electrode without correction. The preferably applied ultrafiltration can be performed according to all suitable methods. Preferably, the ultrafiltration comprising at least one volume exchange with water, preferably at least five volume exchanges with water, more preferably at least 10 volume exchanges with water. According to an embodiment of the present invention, the ultrafiltration does not comprise a volume exchange with an acid. Preferably, the ultrafiltration does not comprise a volume exchange with a base. More preferably, the ultrafiltration does not comprise a volume exchange with an acid and does not comprise a volume exchange with a base.

The purified mixture can be subjected directly, without any further intermediate stage, to step (ii) or optionally to further reducing condition as described hereinunder.

It is also possible to freeze the purified mixture and subject it to further reducing condition as described hereinunder after suitable unfreezing.

According to a preferred embodiment of the invention, in step (i), subsequent to the reductive amination conditions, and the optional work-up described above, the HAS derivative is subjected to further reducing conditions.

As possible reducing agents in this step, complex hydrides such as borohydrides, especially sodium borohydride, and thiols, especially dithiothreitol (DTT) and dithioerythritol (DTE) or phosphines such as tris-(2-carboxyethyl)phosphine (TCEP) are mentioned. The reduction is preferably carried out using borohydrides, especially sodium borohydride. Preferably, the reducing agent is used in an excess, more preferably at a concentration in the range of from 0.02 to 1.5 M, more preferably in the range of from 0.05 to 1 M, most preferably in the range of from 0.1 to 0.5 M with respect to the total volume of the reaction solution. Further, this deprotection step is preferably carried out at a temperature in the range of from 0 to 80° C., more preferably in the range of from 10 to 50° C. and especially preferably in the range of from 15 to 35° C. During the course of the reaction, the temperature may be varied, preferably in the above-given ranges, or held essentially constant.

Preferably, the reaction is carried out in aqueous medium. The term “aqueous medium” as used in this context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), dimethylacetamide (DMA), N-methylpyrrolidinone (NMP), alcohols such as methanol, ethanol or isopropanol, acetonitrile, tetrahydrofurane or dioxane. The aqueous solution may also contain a transition metal chelator (disodium ethylenediaminetetraacetate, EDTA, or the like) in a concentration ranging from 0.01 to 100 mM most preferably from 0.1 to 10 mM, such as about 1 mM. The preferred solvent is water.

The HAS derivative is reacted in the further reduction step preferably at a concentration in the range of from 1% to 30% weight.-%, more preferably in the range of from 5% to 20% weight.-%, most preferably 10% weight.-% based on the total weight of the solution.

Most preferably, in the further reducing step, sodium borohydride (NaBH₄) is employed as reducing agent. Preferably, the mixture obtained from adding the sodium borohydride comprises the sodium borohydride preferably at a concentration in the range of from 0.05 to 1.5 mol/l, more preferably from 0.05 to 1 mol/l, more preferably from 0.1 to 0.5 mol/1. Preferably, in (i), the mixture contains the hydroxyalkyl starch and the hydroxyalkyl starch derivative at a concentration in the range of from 1 to 40 weight-%, more preferably from 5 to 30 weight-%, more preferably from 10 to 20 weight-%. Subjecting the mixture to these further reducing conditions in (i) can be carried out for any suitable time period. Generally, the time period is in the range of from 10 min to 24 h. Preferably, the time period is in the range of from 0.25 to 4 h, more preferably from 1 to 3 h. The further reduction reaction using sodium boohhydrate can be carried out at every suitable temperature. Preferably, the reduction with sodium borohydrate is carried out a temperature in the range of from 5 to 40° C., more preferably from 10 to 35° C., more preferably from 20 to 30° C. such as at room temperature. Preferably, in (i), subjecting the mixture to the further reducing conditions comprises keeping the mixture at a temperature in the range of from 10 to 35° C. for a period of from 0.25 to 4 h, more preferably at a temperature in the range of from 20 to 30° C. for a period of from 1 to 3 h.

According to the embodiment, wherein the HAS derivative according to formula (Ib)

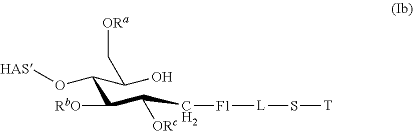

has been subjected to the further reducing step, this HAS derivative may then preferably be isolated from the reaction mixture by any suitable method, such as ultrafiltration or dialysis, preferably ultrafiltration, more preferably followed by lyophilization of the isolated hydroxyalkyl starch derivative. The ultrafiltration or dialysis is preferably carried out under acidic conditions, more preferably at a pH in the range of from 2 to 6, more preferably in the range of from 3 to 5, and/or in the presence of an ion chelator.

Preferably, the acid, if present, is selected from the group consisting of hydrochloric acid, phosphoric acid, trifluoroacetic acid, acetic acid and mixtures of two or more thereof; preferred buffers are selected from the group consisting of acetate, phosphate and citrate buffers. As ion chelators EDTA (ethylenediamine tetraacetic acid), DTPA (diethylene triamine pentaacetic acid) and related compounds may be mentioned.

Most preferably, an acetic acid buffer (in the range of from 0.1 mM to 1 M, more preferably in the range of from 1 to 100 mM, most preferably 10 mM) with pH 4 which more preferably comprises EDTA in the range of from 0.01 to 100 mM (most preferably 10 mM) is used as ultrafiltration/dialysis buffer. More preferably, after removal of the reaction impurities, the ultrafiltration/dialysis buffer is replaced by water in order to remove buffer salts from the product.

Further, the hydroxyalkyl starch derivative according to formula (Ib) may be precipitated from the reaction mixture, in particular by adding an alcohol, preferably 2-propanol. The obtained precipitate may be collected by filtration or centrifugation and further purified using conventional purification protocols, preferably ultrafiltration, dialysis or chromatographic methods, preferably size exclusion chromatography.

### The Group T

As mentioned above, T is H or a thiol protecting group PG.

Thus, the present invention also relates to a method as described above, and a conjugate obtained or obtainable by said method, comprising
(i) reacting hydroxyalkyl starch (HAS) of formula (Ia)

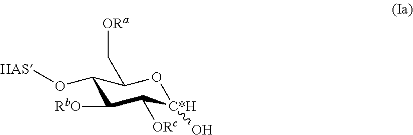

via carbon atom C* of the reducing end of the HAS with the functional group M of a crosslinking compound according to formula (II), thereby obtaining a HAS derivative of one of the following formulas:

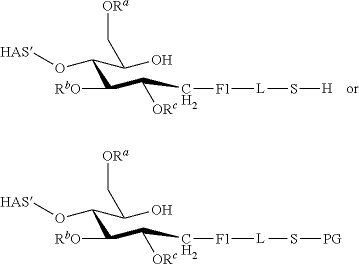

According to a particularly preferred embodiment of the invention, T is H. Thus, the present invention also relates to a method as described above, and a conjugate obtained or obtainable by said method, comprising
(i) reacting hydroxyalkyl starch (HAS) of formula (Ia)

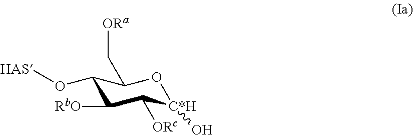

via carbon atom C* of the reducing end of the HAS with the functional group M of a crosslinking compound according to formula (II), thereby obtaining a HAS derivative of the following formula

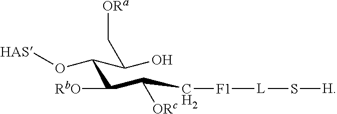

By way of example, the following preferred crosslinking compounds are mentioned: H₂N—CH₂—CH₂—SH, H₂N—CH₂—CH₂—CH₂—SH, H₂N—CH₂—CH₂—CH₂—CH₂—SH, H₂N—CH₂—CH₂—CH₂—CH₂—CH₂—SH, H₂N—CH(COOH)—CH₂—SH, H₂N—CH(COOH)—C(CH₃)₂—SH, H₂N—CH(CH₂OH)—CH₂—SH, H₂N—CH(CH₂OH)—CH₂—CH₂—SH, H₂N—CH(CONH₂)—C(CH₃)₂—SH, H₂N—CH(CONH₂)—CH₂—SH, H₂N—CH(COOH)—CH₂—CH₂—SH, H₂N—CH₂—CH₂—O—CH₂—CH₂—SH, H₂N—CH₂—CH₂—O—CH₂—CH₂—O—CH₂—CH₂—SH, H₂N—CH₂—CH₂—O—CH₂—CH₂—O—CH₂—CH₂—O—CH₂—CH₂—SH, H₂N—CH(COOH)—CH₂—CH₂—C(═O)NH—CH—C(═O)(—NH—CH₂—COOH)—CH₂—SH, H₂N—O—CH₂—CH₂—SH, H₂N—O—CH₂—CH₂—CH₂—SH, H₂N—O—CH₂—CH₂—CH₂—CH₂—SH, H₂N—O—CH₂—CH₂—CH₂—CH₂—CH₂—CH₂—SH, H₂N—O—CH(COOH)—CH₂—SH, H₂N—O—CH(COOH)—C(CH₃)₂—SH, H₂N—O—CH(CH₂OH)—CH₂—SH, H₂N—O—CH(CH₂OH)—CH₂—CH₂—SH, H₂N—O—CH(CONH₂)—C(CH₃)₂—SH, H₂N—NH—CH₂—CH₂—SH, H₂N—NH—CH₂—CH₂—CH₂—SH, H₂N—NH—CH₂—CH₂—CH₂—CH₂—SH, H₂N—NH—CH₂—CH₂—CH₂—CH₂—CH₂—SH, H₂N—NH—CH(COOH)—CH₂—SH, H₂N—NH—CH(COOH)—C(CH₃)₂—SH, H₂N—NH—CH(CH₂OH)—CH₂—SH, H₂N—NH—CH(CH₂OH)—CH₂—CH₂—SH, H₂N—NH—CH(CONH₂)—C(CH₃)₂—SH, H₂N—NH—C(═O)—CH₂—SH, H₂N—NH—C(═O)—CH₂—CH₂—SH, H₂N—NH—C(═O)—CH₂—CH₂—CH₂—SH, H₂N—NH—C(═O)—CH₂—CH₂—CH₂—CH₂—SH, H₂N—NH—C(═O)—CH₂—CH₂—CH₂—CH₂—CH₂—SH, H₂N—NH—C(═O)—CH(COOH)—CH₂—SH, H₂N—NH—C(═O)—CH(COOH)—C(CH₃)₂—SH, H₂N—NH—C(═O)—CH(CH₂OH)—CH₂—SH, H₂N—NH—C(═O)—CH(CH₂OH)—CH₂—CH₂—SH and H₂N—NH—C(═O)—CH(CONH₂)—C(CH₃)₂—SH.

Preferably, the crosslinking compound according to formula (II) is selected from the group consisting of H₂N—CH₂—CH₂—SH, H₂N—CH₂—CH₂—CH₂—SH, H₂N—CH₂—CH₂—CH₂—CH₂—SH, H₂N—CH₂—CH₂—CH₂—CH₂—CH₂—SH, H₂N—CH(COOH)—CH₂—SH and H₂N—CH(COOH)—C(CH₃)₂—SH, H₂N—CH(CH₂OH)—CH₂—SH, H₂N—CH(CH₂OH)—CH₂—CH₂—SH, H₂N—CH(CONH₂)—C(CH₃)₂—SH, H₂N—CH(CONH₂)—CH₂—SH, H₂N—CH(COOH)—CH₂—CH₂—SH, H₂N—CH₂—CH₂—O—CH₂—CH₂—SH, H₂N—CH₂—CH₂—O—CH₂—CH₂—O—CH₂—CH₂—SH, H₂N—CH₂—CH₂—O—CH₂—CH₂—O—CH₂—CH₂—O—CH₂—CH₂—SH, H₂N—CH(COOH)—CH₂—CH₂—C(═O)NH—CH—[C(═O)(—NH—CH₂—COOH)]—CH₂—SH and H₂N—O—CH₂—CH₂—SH.

Most preferably, the crosslinking compound (II) is cysteamine H₂N—CH₂—CH₂—SH.

According to an alternative embodiment of the invention T is PG, wherein PG may be any suitable SH protecting group known to those skilled in the art. Preferably, PG is a protecting group forming together with —S— a thioether (e.g. benzyl, allyl, triarylmethyl groups, such as trityl (Trt)), a disulfide (e.g. S-sulfonates, S-tert-butyl, S-(2-aminoethyl), 2-pyridylthio). In case T is a thiol protecting group PG, step (i) further comprises a deprotection step.

In case the group —S—PG is a disulfide, the crosslinking compound according to formula (II) M-L-S—PG is preferably a symmetrical disulfide, with PG having the structure —S-L-M or is selected from the group consisting of 2-pyridylthio, —S—SO₃—, —S—SO₂-aryl and —S—SO₂-alkyl.

According to a preferred embodiment of the invention, the group S-PG present in the crosslinking compound according to formula (II) is selected from the group consisting of —S-Trt, —S—S-L-M, —S—S-tBu, —S—S-(2-pyridyl), —S—SO₃—, —S—SO₂-aryl and —S—SO₂-alkyl, in particular the group S-PG is —S—S-L-M.

According to this embodiment, the crosslinking compound according to formula (II) is selected from the group consisting of H₂N-L-S-Trt, H₂N-L-S—S-L-NH₂, H₂N-L-S—S-tBu, H₂N-L-S—S-(2-pyridyl), H₂N-L-S—SO₃—, H₂N-L-S—SO₂-aryl and H₂N-L-S—SO₂-alkyl, most preferably the crosslinking compound according to formula (II) is H₂N-L-S—S-L-NH₂.

By way of example, the following particularly preferred crosslinking compounds are mentioned: H₂N—CH₂—CH₂—S-Trt, H₂N—CH₂—CH₂—CH₂—S-Trt, H₂N—CH₂—CH₂—CH₂—CH₂—S-Trt, H₂N—CH₂—CH₂—CH₂—CH₂—CH₂—S-Trt, H₂N—CH₂—CH₂—S—S—CH₂—CH₂—NH₂, H₂N—CH₂—CH₂—S—S-tBu, H₂N—CH₂—CH₂—CH₂—S—S-tBu, H₂N—CH₂—CH₂—CH₂—CH₂—S—S-tBu, H₂N—CH(COOH)—CH₂—S-Trt, H₂N—CH(COOH)—C(CH₃)₂—S-Trt, H₂N—CH(COOH)—CH₂—S—S—CH₂—CH(COOH)—NH₂, H₂N—CH(COOH)—CH₂—CH₂—C(═O)NH—CH—[C(═O)(—NH—CH₂—COOH)]—CH₂—S—S—CH₂—CH—[C(═O)(—NH—CH₂—COOH)]—NH—C(═O)—CH₂—CH₂—CH(COOH)—NH₂. Most preferably, the crosslinking compound (II) is cystamine H₂N—CH₂—CH₂—S—S—CH₂—CH₂—NH₂ or cystine H₂N—CH(COOH)—CH₂—S—S—CH₂—CH(COOH)—NH₂, more preferably cystamine.

The reaction conditions used in the deprotection step are adapted to the respective protecting group used.

According to a preferred embodiment of the invention, the group —S—PG is a disulfide, as described above. In this case, the deprotection step comprises the reduction of this disulfide bond to give the respective thiol group. This deprotection step is preferably carried out using specific reducing agents. As possible reducing agents, complex hydrides such as borohydrides, especially sodium borohydride, and thiols, especially dithiothreitol (DTT) and dithioerythritol (DTE) or phosphines such as tris-(2-carboxyethyl)phosphine (TCEP) are mentioned. The reduction is preferably carried out using borohydrides, especially sodium borohydride. Preferably, the reducing agent is used in an excess, more preferably at a concentration in the range of from 0.02 to 1.5 mol/l, more preferably in the range of from 0.05 to 1 mol/l, most preferably in the range of from 0.1 to 0.5 mol/l with respect to the total volume of the reaction solution. Further, this deprotection step is preferably carried out at a temperature in the range of from 0 to 80° C., more preferably in the range of from 10 to 50° C. and especially preferably in the range of from 15 to 35° C. During the course of the reaction, the temperature may be varied, preferably in the above-given ranges, or held essentially constant.

Preferably, the reaction is carried out in aqueous medium. The term “aqueous medium” as used in this context of the present invention refers to a solvent or a mixture of solvents comprising water in an amount of at least 10% per weight, preferably at least 20% per weight, more preferably at least 30% per weight, more preferably at least 40% per weight, more preferably at least 50% per weight, more preferably at least 60% per weight, more preferably at least 70% per weight, more preferably at least 80% per weight, even more preferably at least 90% per weight or up to 100% per weight, based on the weight of the solvents involved. The aqueous medium may comprise additional solvents like formamide, dimethylformamide (DMF), dimethylsulfoxide (DMSO), alcohols such as methanol, ethanol or isopropanol, acetonitrile, tetrahydrofurane or dioxane. Preferably, the aqueous solution contains a transition metal chelator (disodium ethylenediaminetetraacetate, EDTA, or the like) in a concentration ranging from 0.01 to 100 mM, preferably from 0.1 to 10 mM, such as about 1 mM. The preferred solvent is water.

The HAS derivative is reacted in the reduction step at a concentration in the range of from 1% to 30% weight.-%, more preferably in the range of from 5% to 20% weight.-%, most preferably 10% weight.-% based on the total weight of the solution.

Preferably, the retentate of the ultrafiltration step is directly used for the reduction with NaBH₄.

Thus, the present invention also relates to a method, as described above, and a HAS derivative obtained or obtainable by said method, wherein the removing of the protecting group PG in step (i) is carried out at a temperature in the range of from 0 to 80° C. and in an aqueous solvent system, the group S-PG being a disulfide. The pH value in this deprotection step may be adapted to the specific needs of the reactants for example by using aqueous buffer solutions. Among the preferred buffers, carbonate, phosphate, borate and acetate buffers as well as tris(hydroxymethyl)aminomethane (TRIS) may be mentioned. Preferably in case of sodium borohydride, the reaction is carried out in water at a pH value in the range of 7 to 14. The deprotection step is preferably conducted for a time in the range of from 0.25 to 24 h, more preferably of from 0.5 to 18 h; most preferably of from 0.5 to 4 h.

The conditions for deprotection of thiol groups comprising trityl groups are known to those skilled in the art and are e.g. described in. S. Herman et al., Bioconjugate Chemistry, 1993, 4, 402-405.

For the removal of other protecting groups, literature such as P. G. M. Wuts and T. W. Greene: Protecting Groups in Organic Synthesis, Wiley, 2007, Ch. 6 (p. 647-695), is referenced.

Due the tendency of thiols to form disulfide bridges in solution, the reduction conditions described above with respect to the removal of the protecting group may optionally as well be applied on HAS derivatives with T=H, thus derivatives obtained upon reacting HAS with compound (II), in which T is H, or HAS derivatives obtained after removal of the protecting group PG, in particular after deprotection under non-reductive conditions.

After the reaction with the crosslinking compound according to formula (II) and optionally the deprotection step, at least one isolation step/and or purification step, as described above, may be carried out. The HAS derivative obtained in step (i) may e.g. be isolated from the reaction mixture by any suitable method, such as ultrafiltration or dialysis, preferably ultrafiltration followed by lyophilization of the isolated hydroxyalkyl starch derivative.

In case ultrafiltration or dialysis is carried out, this ultrafiltration or dialysis is preferably performed under acidic conditions, preferably at a pH in the range of from 2 to 6, more preferably in the range of from 3 to 5, and/or in the presence of an ion chelator. Preferably an acid selected from the group comprising hydrochloric acid, phosphoric acid, trifluoroacetic acid and acetic acid is employed. Preferred buffers are selected from the group comprising acetate, phosphate and citrate buffers. As suitable ion chelators EDTA (ethylenediamine tetraacetic acid), DTPA (diethylene triamine pentaacetic acid) and related compounds may be mentioned.

Most preferably, an acetic acid buffer (preferably in the range of from 0.1 mM to 1 M, more preferably in the range of from 1 to 100 mM, most preferably 10 mM) with pH 4 preferably comprising EDTA in the range of from 0.01 to 100 mM (most preferably 1-10 mM) is used as ultrafiltration or dialysis buffer. After removal of the reaction impurities, the ultrafiltration/dialysis buffer is replaced by water in order to remove buffer salts from the product.

Further, the hydroxyalkyl starch derivative may be precipitated from the reaction mixture, in particular by adding an alcohol, preferably 2-propanol. The obtained precipitate may be collected by filtration or centrifugation and further purified using conventional purification protocols, preferably ultrafiltration, dialysis or chromatographic methods, preferably size exclusion chromatography.

Most preferably the obtained derivative is further lyophilized prior to step (ii) until the solvent content of the reaction product is sufficiently low according to the desired specifications of the derivative.

Step (ii) In step (ii) of the invention, the HAS derivative of formula (Ib) obtained in step (i) is reacted with a crosslinking compound of formula (III)

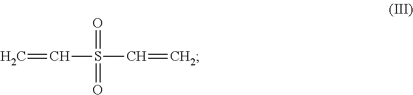

thereby obtaining a HAS derivative of formula (I)

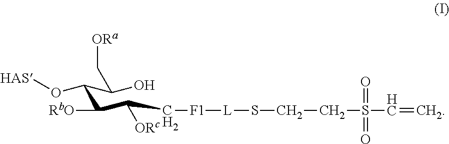

Step (ii) according to the invention is preferably carried out in a solvent selected from the group consisting of DMSO, DMF, DMA, NMP, formamide, water, reaction buffers and mixtures of two or more thereof, more preferably in a mixture of DMSO and reaction buffer. Preferred reaction buffers are, e.g., sodium citrate buffer, sodium acetate buffer, sodium phosphate buffer, sodium carbonate buffer, sodium borate buffer. Preferred pH values of the reaction buffers are in the range of from 2 to 10, more preferably of from 2, 5 to 7, more preferably of from 3 to 5, most preferably at a pH of around 4. Thus, the present invention also relates to a method, as described above, and a HAS derivative obtained or obtainable by said method, wherein the reacting according to step (ii) is carried out at a pH in the range of from 2 to 10, more preferably of from 2.5 to 7, more preferably of from 3 to 5, most preferably at a pH of around 4. The reaction buffer may further contain an ion chelator such as EDTA or DTPA preferably at a concentration between 0.01 and 100 mmol/L, more preferably between 1 and 10 mmol/L.

The crosslinking compound of formula (III) is preferably added as liquid to the aqueous medium.

The reaction mixture is preferably stirred at a temperature in the range of from 0° C. to 50° C., more preferably at a temperature in the range of from 10 to 40° C., more preferably in the range of from 20 to 30° C. During the course of the reaction, the temperature may be varied, preferably in the above-given ranges, or held essentially constant.

Thus, the present invention also relates to a method, as described above, and a HAS derivative obtained or obtainable by said method, wherein the reacting according to step (ii) is carried out at a temperature in the range of from 0° C. to 50° C. and in a solvent selected from the group consisting of DMSO, DMF, DMA, NMP, formamide, water, reaction buffers and mixtures thereof.

The reaction is preferably conducted for a time in the range of from 5 min to 48 h, more preferably from 10 min to 24 h, more preferably from 30 min to 10 h, more preferably from 30 min to 2 h.

The molar ratio of crosslinking compound (III):the thiol content of the HAS derivative (Ic), measured as described in “Instructions Ellman's Reagent, Pierce Biotechnology, Inc. 7/2004, USA” is preferably in the range of from 0.9 to 100, more preferably from 2 to 20, more preferably from 3 to 10, and most preferably 5.

The concentration of the HAS derivative (Ib), in the solvent, preferably the aqueous system, is preferably in the range of from 1 to 50 wt. %, more preferably of from 5 to 40 wt.-%, more preferably of from 10 to 30 wt.-%, and most preferably 20 wt.-%, relating, in each case, to the weight of the reaction solution.

Preferably, the hydroxyalkyl starch derivative (I)

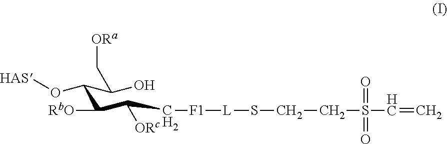

obtained in step (ii), is isolated from the reaction mixture by ultrafiltration or dialysis, preferably ultrafiltration followed by lyophilization or the hydroxyalkyl starch derivative is precipitated form the reaction mixture, in particular by adding an alcohol, preferably 2-propanol or water. The obtained precipitate may be collected by filtration or centrifugation and further purified using conventional purification protocols, preferably ultrafiltration, dialysis or chromatographic methods, preferably size exclusion chromatography.

### Use of the HAS Derivative (I)

The HAS derivative according to formula (I) is preferably used as reactant for coupling to a thiol group comprising compound Q.

The term “thiol group comprising compound Q” as used in the context of the present invention relates to any substance comprising a thiol group, preferably to a substance which can affect any physical or biochemical property of a biological organism including, but not limited to, viruses, bacteria, fungi, plants, animals, and humans. In particular, the term “thiol group comprising compound Q” as used in the context of the present invention relates to a substance intended for diagnosis, cure, mitigation, treatment, or prevention of a disease in humans or animals, or to otherwise enhance physical or mental well-being of humans or animals. Preferred examples of such substances include, but are not limited to, thiol group comprising, peptides, polypeptides, enzymes, small molecule drugs, dyes, lipids, nucleosides, nucleotides, nucleotide analogs, oligonucleotides, nucleic acid analogs, cells, viruses, liposomes, microparticles, and micelles. It is to be understood that any derivatives of the aforementioned terms are included within the meaning of the present invention. The term “derivatives thereof” refers to chemically modified derivatives, mutants, and functional mimetics of the aforementioned compounds. Preferably, a biologically active substance according to the present invention contains a native thiol group. However, such thiol group may also be introduced by methods well known to those skilled in the art.

The term “thiol group comprising peptide” as used in the context of the present invention is denoted to mean peptides comprising up to 50 natural or unnatural, D- or L-amino acids and comprising at least one thiol group. The thiol group may be part of a cysteine or may be introduced into the peptide by a chemical modification.

The term “thiol comprising polypeptide” as used in the context of the present invention includes all compounds having a peptidic backbone and more than 50 monomer (amino acid) units and which comprise at least one thiol group. This term thus in particular includes proteins. The term “protein” as used in the context of the present invention includes natural proteins as well as chemically modified derivatives, mutants and analogs thereof. The term “protein mutant” is denoted to mean a protein being modified with at least one natural or unnatural amino acid either at the N- or at the C-terminus of the protein or in which at least one naturally-occurring amino acid within the sequence is replaced with another natural or unnatural amino acid. It has to be understood that the thiol group may be present in the wild type protein as such or may be introduced by any suitable method such as by adding (e.g. by recombinant means) a cysteine residue either at the N- or at the C-terminus of the polypeptide or by replacing (e.g. by recombinant means) a naturally-occurring amino acid by cysteine to give a mutant of the protein. The respective methods are known to the person skilled in the art (see Elliott, Lorenzini, Chang, Barzilay, Delorme, 1997, Mapping of the active site of recombinant human erythropoietin, Blood, 89(2), 493-502; Boissel, Lee, Presnell, Cohen, Bunn, 1993, Erythropoietin structure-function relationships. Mutant proteins that test a model of tertiary structure, J. Biol. Chem., 268(21), 15983-93)). Further, any group present in the protein may be chemically modified to give a chemically modified derivative of the protein, such as by addition of a suitable linker compound to the N-terminus or C-terminus or to a side chain either during the synthesis of the protein or to the existing full length protein as such.

Preferred examples of peptides include, but are not limited to, peptide hormones and peptide aptamers. Preferred examples of polypeptides or proteins include, but are not limited to, the following proteins, plasma proteins such as immunoglobulins, growth factors, glucagon-like peptides, cytokines, coagulation factors including vWF, enzymes and enzyme inhibitors, albumins and binding proteins such as alternative scaffold proteins, antibody fragments and soluble receptors. The term “alternative scaffold protein” as used in the context of the present invention relates to a molecule having binding abilities similar to a given antibody wherein the molecule is based on an alternative non-antibody protein framework (see e.g. Hey, T. et al., 2005, Artificial, non-antibody binding proteins for pharmaceutical and industrial applications, Trends Biotechnol., 23(10), 514-22).

As used herein, the term “oligonucleotide” or “nucleic acids” refers to polymers, such as DNA and RNA, of nucleotide monomers or nucleic acid analogs thereof, including double and single-stranded deoxyribonucleotides, ribonucleotides, alpha-anomeric forms thereof, and the like. Usually the monomers are linked by phosphodiester linkages, wherein the term “phosphodiester linkage” refers to phosphodiester bonds or bonds including phosphate analogs thereof, including associated counterions, e.g., H⁺, NH₄⁺, Na⁺. The term oligonucleotide further includes polymers comprising mixtures of deoxyribonucleotides and ribonucleotides (DNA/RNA-hybrids). Further the term includes derivatives thereof chemically modified derivative of the oligonucleotides.

“Nucleoside” refers to a compound consisting of a purine, deazapurine, or pyrimidine nucleobase, e.g., adenine, guanine, cytosine, uracil, thymine, deazaadenine, deazaguanosine, and the like, linked to a pentose at the 1-position. When the nucleoside base is purine or 7-deazapurine, the pentose is attached to the nucleobase at the 9-position of the purine or deazapurine, and when the nucleobase is pyrimidine, the pentose is attached to the nucleobase at the 1-position of the pyrimidine.

“Nucleotide” refers to a phosphate ester of a nucleoside, e.g., a triphosphate ester, wherein the most common site of esterification is the hydroxyl group attached to the C-5 position of the pentose. A nucleotide is composed of three moieties: a sugar, a phosphate, and a nucleobase (Blackburn, G. and Gait, M. Eds. “DNA and RNA structure” in Nucleic Acids in Chemistry and Biology, 2nd Edition, (1996) Oxford University Press, pp. 15-81). When part of a duplex, nucleotides are also referred to as “bases” or “base pairs”.

The term “nucleic acid analogs” refers to analogs of nucleic acids made from monomeric nucleotide analog units, and possessing some of the qualities and properties associated with nucleic acids. For example, nucleic acid analogs comprise modifications in the chemical structure of the base (e.g. C-5-propyne pyrimidine, pseudo-isocytidine and isoguanosine), the sugar (e.g. 2′-O-alkyl ribonucleotides) and/or the phosphate (e.g. 3′-N-phosphoramidate). See for example Englisch, U. and Gauss, D. “Chemically modified oligonucleotides as probes and inhibitors”, Angew. Chem. Int. Ed. Engl. 30:613-29 (1991)). Nucleotide analogs in particular include, but are not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halogen, SH, SR, NH₂, NHR, NR₂, or CN, wherein R is an alkyl moiety. Nucleotide analogs are also meant to include nucleotides with other modified bases, or with different sugars such as 2′-methyl ribose as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles. Nucleotide analogs are also meant to include nucleotides with non-natural linkages such as methylphosphonates and phosphorothioates. Further the term is meant to include analogs such as locked nucleic acids in which the ribose moiety of the nucleotide is modified with an extra bridge connecting the 2′-oxygen and 4′-carbon (LNA). Further, the term nucleotide also includes those species that have a detectable label, such as for example a radioactive or fluorescent moiety, or mass label attached to the nucleotide. A class of analogs where the sugar and phosphate moieties have been replaced with a 2-aminoethylglycine amide backbone polymer is peptide nucleic acid (PNA) (Nielsen, P., Egholm, M., Berg, R. and Buchardt, O. “Sequence-selective recognition of DNA by strand displacement with a thymidine-substituted polyamide”, Science 254:1497-1500 (1991)). Further the oligonucleotide according to the invention may comprise one or more abasic sites. By “abasic site” is meant a monomeric unit contained within an oligonucleotide chain but which does not contain a purine or pyrimidine base.

Preferred examples of oligonucleotides and nucleic acid analogs include, but are not limited to, thiol group comprising, ribonucleic acids, deoxyribonucleic acids, peptide nucleic acids (PNA), locked nucleic acids (LNA).

The thiol group present in the oligonucleotides, nucleotides, nucleosides or nucleic acid analogs of the invention may be attached by any method known to those skilled in the art, i.e., for example, either by introducing a thiol modified building block during the preparation of the respective compound or by chemical modification to any suitable position of the respective compound, in particular by attaching a thiol group comprising linker in 3′- or 5′-position.

The term “lipids” refers to a broad group of naturally occurring and unnatural molecules that include fats, waxes, sterols, fat-soluble vitamins (such as vitamins A, D, E, and K), monoglycerides, diglycerides, triglycerides, phospholipids, and others. Lipid compounds are suitable for the transport of biologically active substances or molecules. Polymer conjugated lipid compounds are useful in drug delivery, for example in the form of liposomes. (Immordino et al., IJN, 2006: I (3) 297-315).

Preferably, compound Q is selected from the group consisting of, thiol group comprising, peptides, polypeptides, oligonucleotides and nucleic acid analogs, more preferably from the group consisting of peptide hormones, peptide aptamers, plasma proteins (such as immunoglobulins, growth factors, cytokines, coagulation factors including vWF), glucagon-like-peptides, enzymes, enzyme inhibitors, albumins, natural or artificial binding proteins (such as alternative scaffold proteins, antibody fragments, soluble receptors), ribonucleic acids, deoxyribonucleic acids, peptide nucleic acids (PNA) and locked nucleic acids (LNA).

Further, the present invention relates to a hydroxyalkyl starch (HAS) derivative of formula (IV), as described above, or of a salt or solvate thereof,

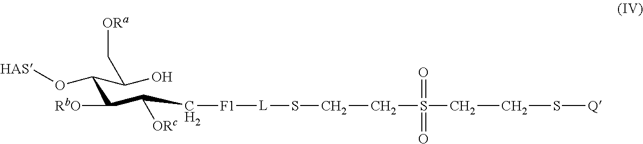

wherein
-Q′ is the remainder of a thiol group comprising compound Q which is linked via the group —S— of the thiol group to the —CH₂— group; and wherein Q selected from the group consisting of, thiol group comprising, peptides, polypeptides, oligonucleotides and nucleic acid analogs, more preferably from the group consisting of peptide hormones, peptide aptamers, plasma proteins (such as immunoglobulins, growth factors, cytokines, coagulation factors including vWF), glucagon-like-peptides, enzymes, enzyme inhibitors, albumins, natural or artificial binding proteins (such as alternative scaffold proteins, antibody fragments, soluble receptors), ribonucleic acids, deoxyribonucleic acids, peptide nucleic acids (PNA) and locked nucleic acids (LNA). Further, the present invention also relates to the use of such HAS derivatives as a medicament.

According to a preferred embodiment of the invention, compound Q is a, thiol group comprising, peptide or polypeptide.

Thus, the present invention also relates to a hydroxyalkyl starch (HAS) derivative of formula (IV) as described above, or a salt or solvate thereof, wherein Q is a, thiol group comprising, peptide or polypeptide. Further, the present invention also relates to the use of such HAS derivatives as a medicament.

Particularly preferred examples of thiol group comprising, peptides and polypeptides (proteins) include, but are not limited to, the following peptides and proteins, or derivatives thereof: erythropoietin (EPO), such as recombinant human EPO (rhEPO) or an EPO mimetic, colony-stimulating factors (CSF), such as G-CSF like recombinant human G-CSF (rhG-CSF), alpha-Interferon (IFN alpha), beta-Interferon (IFN beta) or gamma-Interferon (IFN gamma), such as IFN alpha, IFN beta and IFN gamma like recombinant human IFN alpha, IFN beta or IFN gamma (rhIFN alpha, rhIFN beta or rhIFN gamma), interleukines, e.g. IL-1 to IL-34 such as IL-2 or IL-3 or IL-11 like recombinant human IL-2 or IL-3 (rhIL-2 or rhIL-3), serum proteins such as coagulation factors II-XIII like factor II, factor III, factor V, factor VI, factor VII, factor VIIa, factor VIII, such as full-length FVIII, BDD-FVIII or single-chain FVIII, factor IX, factor X, factor XI, factor XII, factor XIII, von Willebrand factor (vWF), enzymes such as lipases, proteases, peptidases, hydrolases, glycosidases, isomerases, reductases, oxidases, transferases, kinases, phosphatases, serine protease inhibitors such as alpha-1-antitrypsin (A1AT), activated protein C (APC), plasminogen activators such as tissue-type plasminogen activator (tPA), such as human tissue plasminogen activator (hTPA), AT III such as recombinant human AT III (rhAT III), myoglobin, albumins such as human serum albumin (HSA), growth factors, such as epidermal growth factor (EGF), thrombocyte growth factor (PDGF), fibroblast growth factors (FGF), brain-derived growth factor (BDGF), nerve growth factor (NGF), B-cell growth factor (BCGF), brain-derived neurotrophic growth factor (BDNF), ciliary neurotrophic factor (CNTF), transforming growth factors such as TGF alpha or TGF beta, BMPs (bone morphogenic proteins), growth hormones such as human growth hormone (hGH) like recombinant human growth hormone (rhGH), tumor necrosis factors such as TNF alpha or TNF beta, somatostatine, somatotropine, somatomedines, hemoglobin, hormones or prohormones such as insulin, gonadotropin, melanocyte-stimulating hormone (alpha-MSH), triptorelin, hypthalamic hormones such as antidiuretic hormones (ADH and oxytocin as well as releasing hormones and release-inhibiting hormones, parathyroid hormone, thyroid hormones such as thyroxine, thyrotropin, thyroliberin, calcitonin, glucagon, glucagon-like peptides (GLP-1, GLP-2 etc.), exendines such as exendin-4, leptin such as recombinant human leptin (rhLeptin), vasopressin, gastrin, secretin, integrins, glycoprotein hormones (e.g. LH, FSH etc.), melanoside-stimulating hormones, lipoproteins and apo-lipoproteins such as apo-B, apo-E, apo-Lₐ, immunoglobulins such as IgG, IgE, IgM, IgA, IgD and fragments thereof, such as Fab fragments derived from immunoglobuline G molecules (Fab), di-Fabs, tri-Fabs, scFv, bis-scFv, diabodies, triabodies, tetrabodies, minibodies, domain antibodies, v_{H} domain, v_{L} domain, murine immunoglobuline G (mIgG), shark antibodies (IgNAR) and fragments thereof, camelid immunoglobulins and fragments thereof such as v_{HH} domain, receptor proteins, such as cell surface receptors or soluble receptors, hirudin, tissue-pathway inhibitor, plant proteins such as lectin or ricin, bee-venom, snake-venom, immunotoxins, antigen E, alpha-proteinase inhibitor, ragweed allergen, melanin, oligolysine proteins, RGD proteins or optionally corresponding receptors for one of these proteins; prolactin, or an alternative scaffold protein. Particularly preferred examples of polypeptides used as alternative scaffold proteins are derivatives of Protein A, Protein G, lipocalins, CTLA-4, A domain from LDL-receptor like module, ubiquitin, gamma crystallin, repeat proteins such as ankyrin repeat proteins, leucine-rich repeat proteins, tetratricopeptide repeat proteins, HEAT-like proteins, armadillo repeat protein, transferrin, beta-lactamase, C-type lectin domain, fibronectin type III domain 10 proteins, Kunitz domain, knottins such as Ecballium elaterium trypsin inhibitor II (EETI-II) and the C-terminal domain of the human Agouti-related protein (AGRP), tendamistat, thioredoxin, PDZ domain, zinc finger proteins such as the plant homeodomain (PHD) finger protein, T-cell receptors, green-fluorescent protein, Fyn domain 3, Alphabodies, CH2 or CH3 domains of an antibody Fc part.

According to a further particularly preferred embodiment, compound Q is selected from the group consisting of insulin, glucagon, glucagon-like peptides, gastric inhibitory peptides, exendins, ghrelin, PYY and peptide aptamers. It is again to be understood that the term oligonucleotide or nucleic acid, such as a DNA or RNA aptamer is denoted to mean thiol comprising derivatives of these compounds, as already described above. Such thiol modifications are known to those skilled in the art.

Thus, the present invention also relates to a hydroxyalkyl starch (HAS) derivative of formula (IV) as described above, or a salt or solvate thereof, wherein compound Q is an oligonucleotide or nucleic acid, such as a DNA or RNA aptamer. Further, the present invention also relates to the use of such HAS derivatives as a medicament.

According to a further particularly preferred embodiment, compound Q is a growth factor or a cytokine, preferably selected from the group consisting of erythropoietin (EPO), such as recombinant human EPO (rhEPO), colony-stimulating factors (CSF), such as G-CSF like recombinant human G-CSF (rhG-CSF), alpha-Interferon (IFN alpha), beta-Interferon (IFN beta), and gamma-Interferon (IFN gamma), such as recombinant human IFN alpha or IFN beta (rhIFN alpha or rhIFN beta), fibroblast growth factors (FGF), human growth hormone (hGH) like recombinant human growth hormone (rhGH), BMPs (bone morphogenic proteins), interleukines, tumor necrosis factors such as TNF alpha and TNF beta.

According to a further particularly preferred embodiment, compound Q is a protein hormone, preferably selected from the group consisting of leptins, follicle stimulating hormon (FSH) and luteinizing hormon (LH).

According to a further particularly preferred embodiment, compound Q is an enzyme or enzyme inhibitor, preferably selected from the group consisting of alpha-1-antitrypsin (A1AT), antithrombin such as AT III, glucocerebrosidase, acid maltase, alpha-galactosidase, iduronidase, iduronate-2-sulfatase, arylsulfatase B, asparaginase, phenylalanine ammonia-lyase, and L-methioninase.

According to a further particularly preferred embodiment, compound Q is coagulation factor or a protein involved in hemostasis, preferably selected from the group consisting of factor II, factor III, factor V, factor VI, factor VII, factor VIIa, factor VIII, factor IX, factor X, factor XI, factor XII, factor XIII, von Willebrand factor, tissue factor pathway inhibitor (TFPI) and Protein C such as APC.

According to a further particularly preferred embodiment, compound Q is an immunoglobulin or fragment thereof, preferably selected from the group consisting of IgG, Fab fragments, Fc fragments, scFvs and dAbs.

According to a further particularly preferred embodiment, compound Q is an artificial binding protein or alternative scaffold protein, preferably selected from the group consisting of ubiquitin, Protein A, lipocalins, transferrin, fibronectins and soluble receptors.

According to a further particularly preferred embodiment, compound Q is a glucagon-like peptide, preferably GLP-1 or GLP-2.

In another particularly preferred embodiment, compound Q is an oligonucleotide or nucleic acid, such as a DNA or RNA aptamer.

According to a further particularly preferred embodiment, compound Q is selected from the group consisting of ribonucleic acid, deoxyribonucleic acid, peptide nucleic acid (PNA), locked nucleic acid (LNA), antisense RNA, RNAi, siRNA, Spiegelmer, aptamer, ribozyme and phosphorothioate-modified nucleic acid.

Step (iii)

In case the HAS derivative according to formula (I) is reacted with a thiol group comprising compound Q, the method according to the invention further comprises
(iii) reacting the HAS derivative of formula (I) via the group —CH═CH₂ with an —SH group of a thiol group comprising compound Q, thereby forming a HAS derivative of formula (IV)

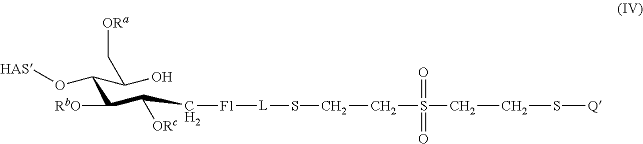

wherein
-Q′ is the remainder of the thiol group comprising compound Q which is linked via the group —S— of the thiol group to the —CH₂ group.

It has surprisingly been found that when conducting the above described method, the reaction product of the hydroxyalkyl starch derivative as obtained in step (ii) with the further compound can be obtained in a high yield and in a high purity. Further, the respective derivative according to formula (IV) shows advantageous properties in terms of stability.

The solvent is chosen depending on the nature of the compound Q to be coupled.

In case Q is a polypeptide, protein or derivative thereof, the reaction is preferably carried out in a solvent selected from the group consisting of water, reaction buffers, DMSO, DMF, DMA, NMP, formamide, and mixtures of two or more thereof.

Preferably the reaction is carried out in an aqueous medium. The term “aqueous medium” is denoted to mean a solvent comprising water and/or at least one reaction buffer. Preferably, the solvent comprises only minor amounts of organic solvents such as in an amount in the range of from 0 to 10% by weight, preferably 0 to 5% by weight, more preferably 0 to 2% by weight, most preferably less than 1% by weight, based on the total weight of the reaction solvent. Further the reaction solvent may comprise detergents, stabilizers, antioxidants and/or reducing agents, preferably at least one antioxidant and/or at least one reducing agent to avoid oxidation of the free thiol groups. A suitable reducing agent may be TCEP, which does not contain a free thiol group and thus does not compete with the Q in the conjugation reaction. A suitable antioxidant may be EDTA, which acts in an indirect manner by complexing transition metal ions, that can catalyze peroxide formation. Preferably, the reaction is carried out in the presence of TCEP and/or EDTA.

According to one preferred embodiment, according to which, Q is a polypeptide, protein or derivative thereof, the polypeptide, protein or derivative thereof is preferably incubated with at least one reducing agent and optionally at least one antioxidant, more preferably with DTT, DTE, beta-mercaptoethanol or TCEP, most preferably with DTT and TCEP prior to the addition to or of the HAS derivative of formula (I). Thiol-containing reducing agents should be carefully removed from the reduced protein by methods known to those skilled in the art to prevent unwanted quenching of the thiol-reactive hydroxyalkyl starch derivative.

Preferred reaction buffers are, e.g., sodium citrate buffer, sodium acetate buffer, sodium phosphate buffer, sodium carbonate buffer, sodium borate buffer and TRIS (tris(hydroxymethyl)aminomethane). Preferred pH values of the reaction buffers are in the range of from 2 to 11, more preferably of from 3 to 10, more preferably of from 7 to 10, and more preferably of from 8 to 9.

The reaction mixture is preferably stirred at a temperature in the range of from 0° C. to 50° C., more preferably at a temperature in the range of from 5 to 40° C., more preferably in the range of from 5 to 25° C. During the course of the reaction, the temperature may be varied, preferably in the above-given ranges, or held essentially constant.

Thus, the present invention also relates to a method, as described above, and a HAS derivative obtained or obtainable by said method, wherein the reacting according to step (iii) is carried out at a temperature in the range of from 0° C. to 50° C. and in a solvent selected from the group consisting of water, reaction buffers, DMSO, DMF, NMP, DMA; formamide, and mixtures of two or more thereof.

The reaction is preferably conducted for a time in the range of from 5 min to 48 h, more preferably of from 20 min to 24 h, more preferably of from 30 min to 18 h.

The molar ratio of compound Q:HAS derivative (I) is preferably in the range of from 1:0.5 to 1:100, more preferably of from 1:1 to 1:20, more preferably of from 1:1 to 1:5 and more preferably of from 1:1.5 to 1:3.

The concentration of the HAS derivative (I) in the solvent, preferably the aqueous system, is preferably in the range of from 0.1 to 50 wt.-%, more preferably from 1 to 30 wt.-%, and more preferably from 1 to 10 wt.-%, relating, in each case, to the weight of the reaction solution.

The term “derivatives of formula (IV)” or “conjugates” of formula (IV) as used hereinunder and above includes the respective pharmaceutically acceptable salts and solvates of these derivatives.

Preferably, the derivatives of formula (IV) described hereinunder and above are at least stable at a pH in range of from 3 to 9, preferably in the range of from 4 to 8, more preferably at a pH in the range of from 4 to 7, more preferably in the range of from 4 to 5.5.

The term “stable” is denoted to mean that the percent of degradation of 1 mg of the derivative in 1 mL buffer solution of a respective pH measured after 20 days of incubation at 40° C., measured by RP-HPLC as described in example C1 is less than 15.5%, more preferably less than 7%, more preferably less than 4%, more preferably less than 2%.

In the following, particularly preferred embodiments of the invention are described:
1. A hydroxyalkyl starch (HAS) derivative of formula (I)

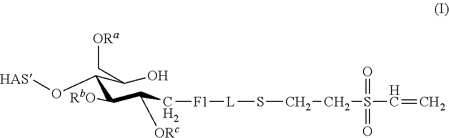

wherein
F1 is a functional group comprising the group —NR′—, with R′ being selected from the group consisting of H, alkyl and acetyl;
L is a spacer bridging F1 and S;
HAS′ is the remainder of the HAS molecule and R^{b} and Re are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6.
2. A hydroxyalkyl starch (HAS) derivative of formula (IV)

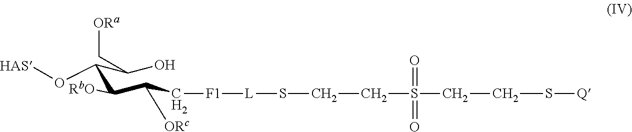

wherein
Q′ is the remainder of a thiol group comprising compound Q which is linked via the group —S— of the thiol to the —CH₂ group;
F1 is a functional group comprising the group NR′—, with R′ being selected from the group consisting of H, alkyl and acetyl;
L is a spacer bridging F1 and S;
HAS′ is the remainder of the HAS molecule and R^{b} and Re are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6.
3. The HAS derivative of embodiment 3, wherein Q is selected from the group consisting of peptides, polypeptides, proteins, enzymes, small molecule drugs, dyes, nucleosides, nucleotides, oligonucleotides, polynucleotides, nucleic acids including peptide nucleic acids, cells, viruses, liposomes, microparticles, micelles or derivatives thereof.
4. The HAS derivative of any one of embodiments 1 to 3, wherein the HAS is hydroxyethyl starch (HES), and R¹, R², R³, and R⁴ are hydrogen, and wherein
   m is 2;
   n is 0 to 4.
5. The HAS derivative of any one of embodiments 1 to 4, wherein F1 is selected from the group consisting of —NH—, —NH—NH—, —NH—NH—C(═O)— and —NH—O—, wherein F is preferably —NH—.
6. The HAS derivative of any one of embodiments 1 to 5, wherein the spacer L comprises, preferably consists of the moiety —(C(L′L″))_{q}- with L′ and L″ in each repeating unit CL′L″ with L′ and L″ in each repeating unit —C(L′L″)- being, independently of each other, selected from the group consisting of H, alkyl, aryl, alkenyl, alkynyl, hydroxyl, alkylcarbonyloxy, arylcarbonyloxy, alkoxycarbonyloxy, aryloxycarbonyloxy, amide, carboxyl, alkoxycarbonyl, aminocarbonyl, alkylaminocarbonyl, dialkylaminocarbonyl, alkoxy, phosphate, phosphonato, phosphinato, acylamino, including alkylcarbonylamino, arylcarbonylamino, carbamoyl, ureido, nitro, alkylthio, arylthio, sulfate, alkylsulfinyl, sulfonate, sulfamoyl, sulfonamido, trifluoromethyl, cyano, azido, carboxymethylcarbamoyl [i.e. the group —C(═O)(—NH—CH₂—COOH)], cycloalkyl such as e.g. cyclopentyl or cyclohexyl, heterocycloalkyl such as e.g. morpholino, piperazinyl or piperidinyl, alkylaryl, arylalkyl and heteroaryl, wherein the groups L′ and L″ in each repeating unit may be the same or may differ from each other, with q preferably being in the range of from 1 to 20, more preferably in the range of from 1 to 10, more preferably in the range of from 2 to 6, more preferably, 2, 3 or 4.
7. The HAS derivative of any one of embodiments 1 to 6, wherein the spacer L is —CH₂—CH₂—.
8. A method for the preparation of a hydroxyalkyl starch derivative comprising
   (i) reacting hydroxyalkyl starch (HAS) of formula (Ia)

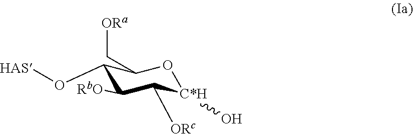

via carbon atom C* of the reducing end of the HAS with the functional group
M of a crosslinking compound according to formula (II)

M-L-S-T (II)
wherein
M comprises the group —NHR′, with R′ being selected from the group consisting of H and alkyl;
L is a spacer bridging M and S;
T is H or a thiol protecting group PG;
HAS′ is the remainder of the HAS molecule and R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are independently hydrogen or an alkyl group having from 1 to 4 carbon atoms, m is 2 to 4, wherein R¹ and R² are the same or different from each other in the m groups CR¹R²; n is from 0 to 6, thereby obtaining a HAS derivative of formula (Ib)

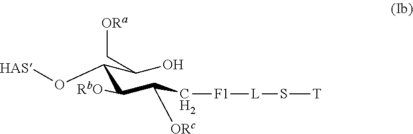

wherein —CH₂—F1- is the moiety resulting from the reaction of the group M with the HAS via the carbon atom C* of the reducing end, and F1 is a functional group comprising the group —NR′—; optionally removing PG in case T is PG to give T=H;
(ii) reacting the HAS derivative of formula (Ib) with a crosslinking compound of formula (III)

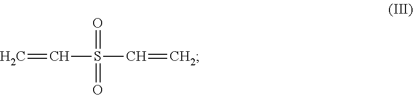

thereby obtaining a HAS derivative of formula (I)

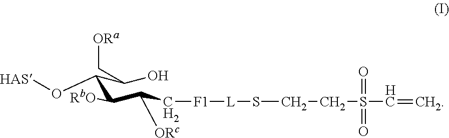

9. The method of embodiment 8, wherein T is a thiol protecting group PG, and wherein step (i) further comprises removing PG from the HAS derivative (Ib).
10. The method of embodiment 8 or 9, wherein PG has the structure —S-L-M or -Trt (Trityl), preferably S-L-M.
11. The method of any one of embodiments 8 to 10, wherein HAS is hydroxyethyl starch (HES),
   and R¹, R², R³, and R⁴ are hydrogen, and wherein
   m is 2;
   n is 0 to 4.
12. The method of any one of embodiments 8 to 11, wherein M is selected from the group consisting of H₂N—, H₂N—NH—, H₂N—NH—C(═O)— and H₂N—O—, M preferably being H₂N—.
13. The method of any one of embodiments 8 to 12, wherein the protecting group PG has the structure —S-L-M.
14. The method of any one of embodiments 8 to 13, wherein the reacting according to (i) is carried out under reductive amination conditions, preferably at a temperature 5° C. to 100° C. and in a solvent selected from the group consisting of DMSO, DMF, DMA, NMP, water, formamide, buffers and mixtures of two or more thereof.
15. The method of any one of embodiments 8 to 14, wherein the spacer L comprises, preferably consists of the moiety —(C(L′L″))_{q}- with L′ and L″ in each repeating unit CL′L″ with L′ and L″ in each repeating unit —C(L′L″)- being, independently of each other, selected from the group consisting of H, alkyl, aryl, alkenyl, alkynyl, hydroxyl, alkylcarbonyloxy, arylcarbonyloxy, alkoxycarbonyloxy, aryloxycarbonyloxy, amide, carboxyl, alkoxycarbonyl, aminocarbonyl, alkylaminocarbonyl, dialkylaminocarbonyl, alkoxy, phosphate, phosphonato, phosphinato, acylamino, including alkylcarbonylamino, arylcarbonylamino, carbamoyl, ureido, nitro, alkylthio, arylthio, sulfate, alkylsulfinyl, sulfonate, sulfamoyl, sulfonamido, trifluoromethyl, cyano, azido, carboxymethylcarbamoyl [i.e. the group —C(═O)(—NH—CH₂—COOH)], cycloalkyl such as e.g. cyclopentyl or cyclohexyl, heterocycloalkyl such as e.g. morpholino, piperazinyl or piperidinyl, alkylaryl, arylalkyl and heteroaryl, wherein the groups L′ and L″ in each repeating unit may be the same or may differ from each other, with q preferably being in the range of from 1 to 20, more preferably in the range of from 1 to 10, more preferably in the range of from 2 to 6, more preferably, 2, 3 or 4.
16. The method of any one of embodiments 8 to 15, wherein the spacer L is —CH₂—CH₂—.
17. The method of any one of embodiments 8 to 16, wherein PG has the structure —S-L-M and wherein the removing of the protecting group PG according to (i) is carried out at a temperature in the range of from 0 to 80° C. and in an aqueous solvent system.
18. The method of any one of embodiments 8 to 17, wherein the reacting according to step (ii) is carried out at a temperature in the range of from 0° C. to 50° C. and in a solvent selected from the group consisting of DMSO, DMF, NMP, DMA, formamide, water, reaction buffers and mixtures of two or more thereof.
19. The method of any one of embodiments 8 to 18, wherein the reacting according to (ii) is carried out at a pH in the range of from 2 to 10, more preferably of from 3 to 5, most preferably at a pH of around 4.
20. The method according to embodiment 14, wherein subsequent to the reductive amination conditions, the HAS derivative is purified, preferably by ultrafiltration, and optionally subjected to further reducing conditions prior to step (ii), in particular by employing NaBH₄.
21. The method of any one of embodiments 8 to 20, further comprising
   (iv) reacting the HAS derivative of formula (I) via the group —CH═CH₂ with an —SH group of a thiol group comprising compound Q, thereby forming a HAS derivative of formula (IV)

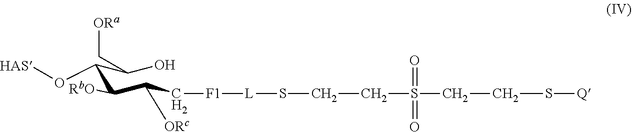

wherein
—S-Q′ Q′ is the remainder of the thiol group comprising compound Q which is linked via the group —S— of the thiol group to the —CH₂ group.
22. The method of embodiment 21, wherein Q is selected from the group consisting of peptides, polypeptides, proteins, enzymes, small molecule drugs, dyes, lipids, nucleosides, nucleotides, oligonucleotides, polynucleotides, nucleic acids including peptide nucleic acids, cells, viruses, liposomes, microparticles, micelles and derivatives thereof.
23. The method of embodiment 21, wherein Q is selected from the group consisting of, thiol group comprising, peptides, polypeptides, oligonucleotides and nucleic acid analogs, more preferably from the group consisting of peptide hormones, peptide aptamers, plasma proteins (such as immunoglobulins, growth factors, cytokines, coagulation factors including vWF), glucagon-like-peptides, enzymes, enzyme inhibitors, albumins, natural or artificial binding proteins (such as alternative scaffold proteins, antibody fragments, soluble receptors), ribonucleic acids, deoxyribonucleic acids, peptide nucleic acids (PNA) and locked nucleic acids (LNA).
24. The method of any one of embodiments 21 to 23, wherein Q is a peptide, polypeptide, protein or derivative thereof, and wherein the reacting according to (iii) is carried out at a temperature in the range of from 0° C. to 50° C. and in a solvent selected from the group consisting of DMSO, DMF, DMA, NMP, water, formamide, reaction buffers and mixtures of two or more thereof.
25. A HAS derivative obtainable or obtained by a method according to any one of embodiments 8 to 20.
26. A HAS derivative, or a salt or solvate thereof, obtainable or obtained by a method according to any one of embodiments 21 to 24.
27. A HAS derivative according to embodiment 26, wherein the derivative is at least stable at a pH in the range of from 3 to 9, preferably in the range of from 4 to 8, more preferably at a pH in the range of from 4 to 7, more preferably in the range of from 4 to 5.5.
28. A HAS derivative according to embodiment 26 or 27, wherein Q is a, thiol group comprising, peptide or polypeptide.
29. A HAS derivative according to embodiment 26 or 27, wherein Q is a glucagon-like peptide, preferably GLP-1 or GLP-2.
30. A HAS derivative according to embodiment 26 or 27, wherein Q is a, thiol group comprising, oligonucleotide or nucleic acid, such as a modified DNA or RNA aptamer.
31. Use of a HAS derivative according to any one of embodiments 1, 4 to 6, or 21 as reactant for coupling to a thiol group of a thiol group comprising compound Q.
32. A HAS derivative as according to any one of embodiments 26 to 30, or salt or solvate thereof, for use as a medicament.

### FIGURES

FIG. 1: Cation exchange chromatography of an L12-HES-Ubi conjugate

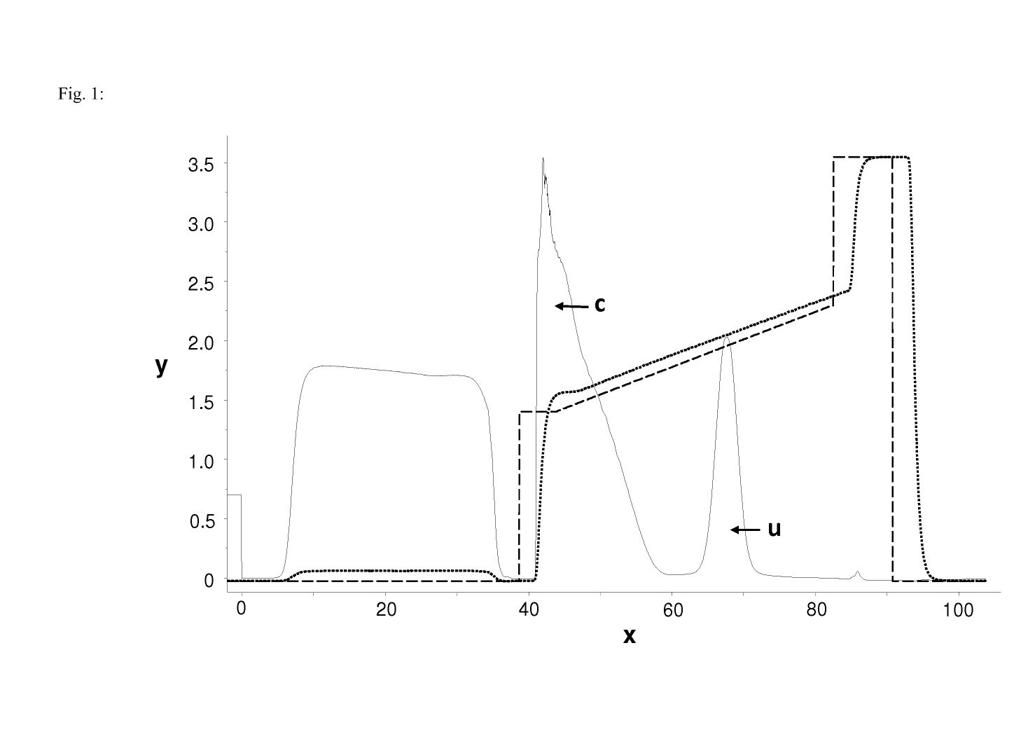

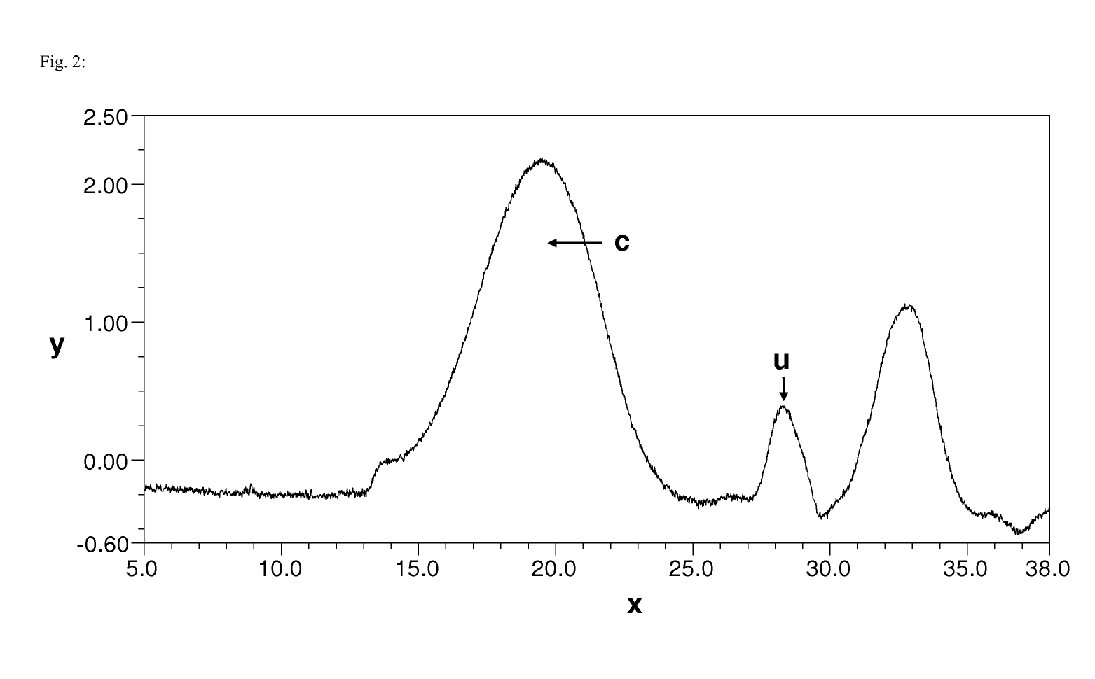

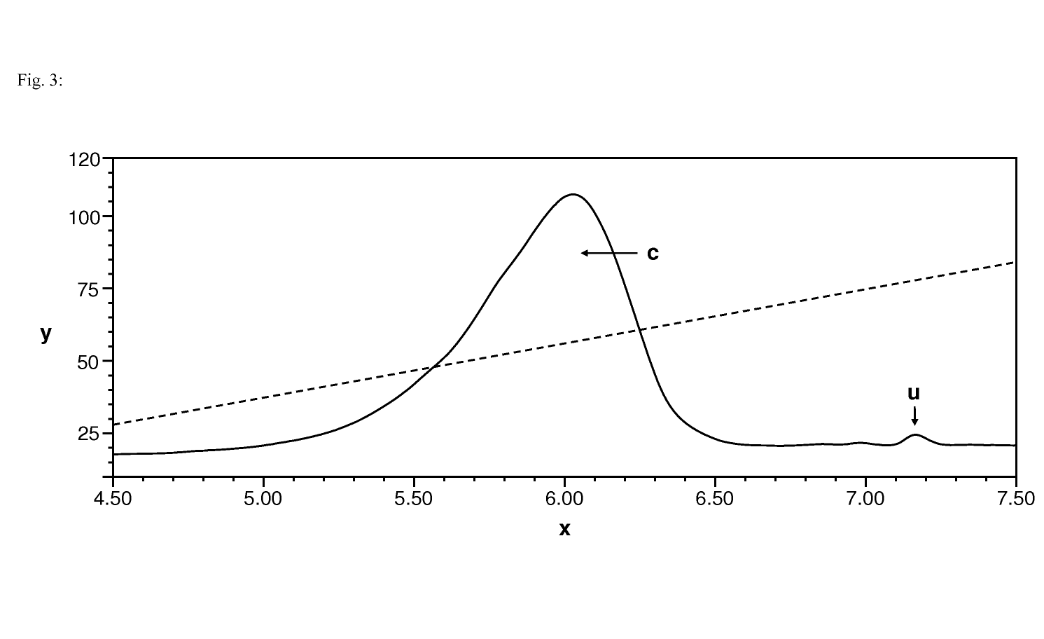

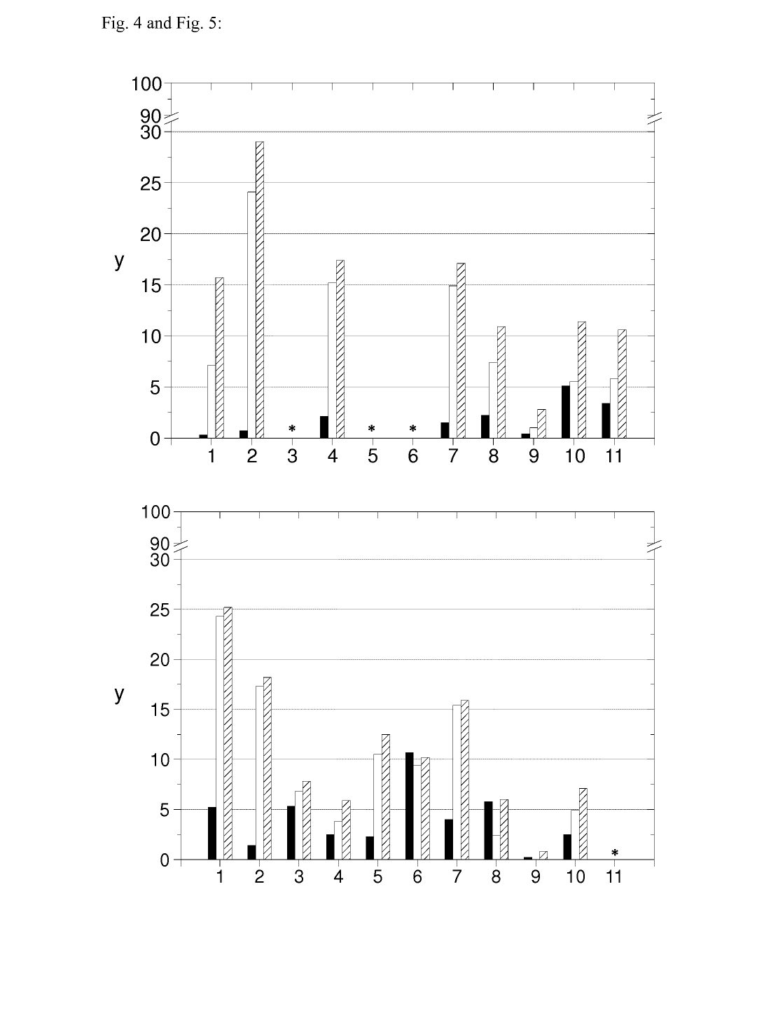

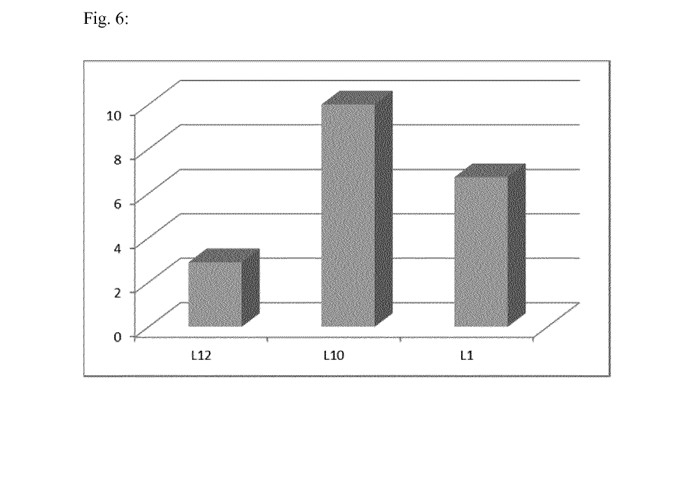

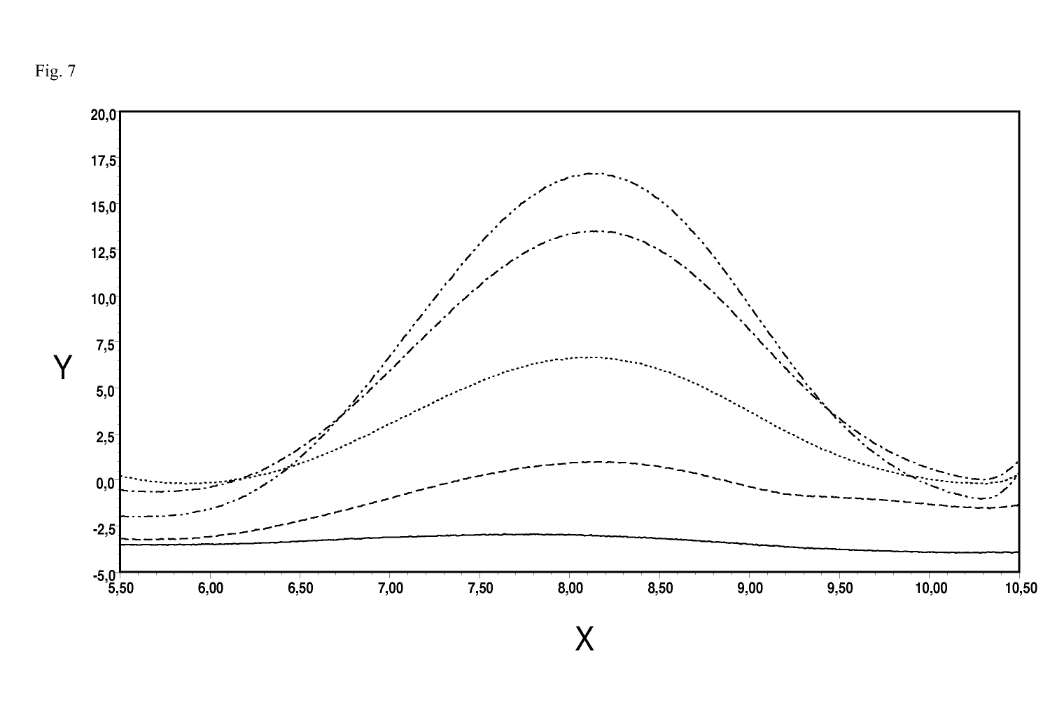

The chromatographic separation of the L12-HES-Ubi coupling reaction according to entry 23, table 4 is monitored by UV spectroscopy at 220 nm (continuous line, y=absorbance in AU) as function of the elution volume (x=elution volume in ml). The percentage of eluent B (broken line) and the conductivity (dotted line, 100% eluent B=86 mS/cm) are shown.

Chromatographic conditions were as follows:

Chromatography system: Äkta Purifier 100 (GE Healthcare)

Column: Hi Trap SP HP 1 ml (GE Healthcare)

Eluent A: 20 mM acetate, pH 4.0

Eluent B: 20 mM acetate, pH 4.0, 1 M NaCl

Operating conditions: flow rate 1.5 ml/min, 25° C.

Gradient: equilibration, 5 CV, 0% B; sample load; wash, 5 CV, 0% B; elution conjugate, 5 CV, 40% B; elution free Ubi, 40 CV, 40-65% B; regeneration, 5 CV, 100% B; reequilibration, 5 CV, 0% B.

Sample load: conjugate 20fold diluted in eluent A and adjusted to pH 4.0

Non-reacted HES derivative is found in the flow-through. The conjugation to the HES derivative weakens the interaction of the protein with the column material resulting in a decrease of elution time for the L12-HES-Ubi conjugate (c) as compared to the unmodified Ubi (u).

FIG. 2: SEC analysis of the coupling reaction of L12-HES to Ubi

FIG. 2 shows a section of an SEC analysis of an L12-HES-Ubi conjugate that was prepared according to example C1. The separation is monitored by UV spectroscopy at 280 nm (y=absorbance in mAU) as function of time (x=retention time in min).

Chromatographic conditions were as follows:

Chromatography system: Ultimate 3000 HPLC System (Dionex)

Column: Superose 12 10/300 GL (GE Healthcare)

Eluent: 1×PBS (5 mM sodium phosphate, 1.7 mM potassium phosphate, 150 mM sodium chloride, pH 7.4, Lonza)

Operating conditions: flow rate 0.5 ml/min, 25° C., run time 60 min

Sample load: 10 μg of reaction mixture, diluted in elution buffer to a final protein concentration of 0.33 g/l

The L12-HES-Ubi conjugate (c) elutes with a retention time of 19.5 min and is separated from the free Ubi (u) eluting at 28.3 min.

FIG. 3: Long time stability study of an L12-HES-Ubi conjugate analyzed by RP-HPLC

FIG. 3 shows a section of a SEC analysis of an L12-HES-Ubi conjugate prepared according to example C1, and table 4, entry 23 and incubated for 20 days at pH 7.0 and 40° C. The chromatographic separation is monitored by UV spectroscopy at 220 nm (continuous line, y=absorbance in mAU) as function of time (x=retention time in min). The section shows a part of the gradient from 27.4% to 32.7% of eluent B (broken line).

Chromatographic conditions were as follows:

Chromatography system: Ultimate 3000 HPLC System (Dionex)

Column: Jupiter C18, 300 Å, 5 μm, 4.6×150 mm (Phenomenex)

Guard column: C18 guard cartridges (Phenomenex) in a SecurityGuard™ Cartridge System (Phenomenex)

Eluent A: 0.1% trifluoroacetic acid in water

Eluent B: 0.1% trifluoroacetic acid in acetonitrile

Operating conditions: flow rate 2 ml/min, 25° C.

Gradient: 0-1.5 min, 2-25% eluent B; 1.5-7 min, 25-35% eluent B; 7-11.5 min, 35-95% eluent B; 11.5-12 min, 95-2% eluent B; 12-13.5 min, 2% eluent B

Sample load: 10 μg of reaction mixture, diluted in demineralized water to a final protein concentration of 0.1 g/l

The L12-HES-Ubi conjugate (c) elutes with a retention time of 6.03 min and is separated from the free Ubi (u) eluting at 7.17 min.

FIG. 4:

Stress stability of Ubi conjugates depending on the linker. The stress stability of various conjugates was determined by RP-HPLC or SEC (see Example C1) after 20 days incubation and is shown as percent of conjugate degradation (y-axis) at pH 4.0 (black bars), pH 7.0 (white bars) and pH 8.0 (broken bars) and at 40° C. X-axis: 1: L1-HES-Ubi, 2: L2-HES-Ubi, 3: L3-HES-Ubi, 4: L4-HES-Ubi, 5: L5-HES-Ubi, 6: L6-HES-Ubi, 7: L10-HES-Ubi, 8: L11-HES-Ubi, 9: L12-HES-Ubi, 10: L13-HES-Ubi, 11: L14-HES-Ubi. Stars indicate Ubi conjugates that were not investigated for stress stability analysis.

This figure clearly demonstrates the higher stability of the tested Ubi comprising conjugates according to the invention at various pH values when compared to the conjugates not according to the invention.

FIG. 5:

Stress stability of MEP conjugates depending on the linker. The stress stability of various conjugates was determined by RP-HPLC (see Example E11) after 20 days incubation and is shown as percent of conjugate degradation (y) at pH 4.0 (black bars), pH 7.0 (white bars) and pH 8.0 (broken bars) and at 40° C. X-axis: 1: L1-HES-MEP, 2: L2-HES-MEP, 3: L3-HES-MEP, 4: L4-HES-MEP, 5: L5-HES-MEP, 6: L6-HES-MEP, 7: L10-HES-MEP, 8: L11-HES-MEP, 9: L12-HES-MEP, 10: L13-HES-MEP, 11: L14-HES-MEP. Stars indicate MEP conjugates that were not investigated for stress stability analysis.

This figure clearly demonstrates the higher stability of the tested MEP comprising conjugates according to the invention at various pH values when compared to the conjugates not according to the invention.

FIG. 6:

Average decomposition rate for conjugates with 3 linkers attached to HES molecules of differing size and different target molecules.

The linker structures L1, L10 and L12 (see Table 1) were attached to thiol-modified HES molecules of different size (Mw ˜30, 100 and 250 kDa, see Table 2). Conjugates with MEP, Ubi and HSA (only largest HES) were prepared and subjected to stress stability as described in examples E11 and C6 (see Table 6, for Ubi; table 6 for HSA). The average degradation rate in % after 20 days for all conjugates of the respective linker tested is shown in the figure.

This figure clearly demonstrates the high stability of the shown conjugates according to the invention at various pH values when compared to shown conjugates not according to the invention.

FIG. 7: Results of size exclusion chromatography according to example E13 Only the relevant section of the chromatograms at the elution time of HES is shown at a wavelength of 220 nm (line solid: chromatogram of HES after mock incubation without modification reagent; line dashed: chromatogram of reaction according to example E13 A (4:1 ratio); line dotted: chromatogram of reaction according to example E13 B (8:1 ratio); line dash-dot: chromatogram of reaction according to example E13 C (20:1 ratio), line dash-dot-dot: chromatogram of reaction according to example E13 D (40:1 ratio).

The following examples are intended to illustrate the present invention without limiting it.

### EXAMPLES

The following abbreviations were used:
A1AT al-antitrypsin
Ac-R6R-NH₂ Ac-Arg-Ser-Cys-Arg-Trp-Arg-NH₂, NeoMPS, France
AU arbitrary unit
CV column volume
DMF N,N-dimethylformamide
DMSO dimethylsulfoxide
DTT dithiothreitol
EDTA ethylenediaminetetraacetic acid disodium salt dihydrate
HES hydroxyethyl starch
HSA human serum albumin
IL1RA interleukin-1 receptor antagonist
MWCO molecular weight cut-off
- none
n.d. not determined
Pierce today Thermo Fisher Scientific
R⁶R H-Arg-Ser-Cys-Arg-Trp-Arg-OH, NeoMPS, France
RGC reactive group content: defined as percentage of HES molecules modified with a certain functional group with respect to all HES molecules (based on Mₙ of the respective HES derivative)
RP-HPLC reversed phase high performance liquid chromatography
SDS-PAGE sodium dodecyl sulfate polyacrylamide gel electrophoresis
SEC size exclusion chromatography
SlyD sensitive to lysis protein D
TFA trifluoroacetic acid
TFE 2,2,2-trifluoroethanol
Ubi Ubiquitin F45W S57C
PET 4-pyridineethanethiol hydrochloride, TCI

All chemicals were obtained from Sigma-Aldrich, Taufkirchen, Germany unless otherwise noted.

### I: Homobifunctional Linker Compounds

The structures of bifunctional linker compounds used in the following examples are given in table 1.

### Example E1

### Synthesis of Linker (L1)

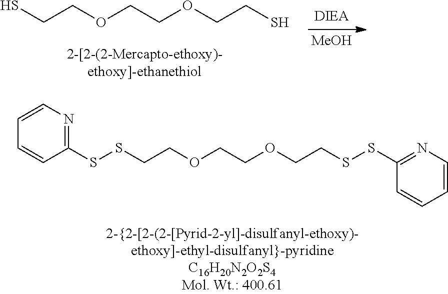 [SMILES: "MeOH" ; SCCOCCOCCS ; c1ccc(SSCCOCCOCCSSc2ccccn2)nc1]

A 1 l three-neck flask was equipped with pressure exchange, magnetic stirring bar and dropping funnel. The flask was loaded with 15.1 g of 2,2′-dithiodipyridine, 500 ml of methanol and 50 μl of N,N-diisopropylethylamine under inert atmosphere. A solution of 2-[2-(2-mercapto-ethoxy)-ethoxy]-ethanethiol in methanol (2 g/120 ml) was added drop-wise over a period of 30 min. 1 h after the ending of the first addition a second portion of the 2-[2-(2-mercapto-ethoxy)-ethoxy]-ethanethiol (0.5 g in 30 ml methanol) was added drop-wise and the reaction mixture was stirred at room temperature.

After complete conversion of the disulfide, the solvent was removed at room temperature under reduced pressure. The residual oil (18.4 g) was purified by flash chromatography on silica (mobile phase: hexane/ethyl acetate 2:1 (v/v)). The crude product was further purified by a second chromatography on silica (mobile phase: hexane/ethyl acetate 1:1 (v/v)). L1 was obtained in 49% yield (2.7 g).

### Example E2

### Synthesis of Linker (L6)

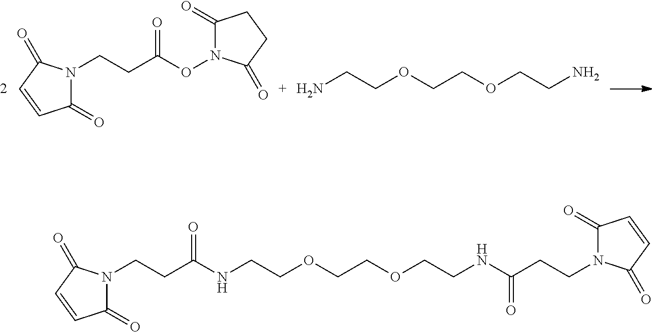 [SMILES: O=C(CCN1C(=O)C=CC1=O)ON1C(=O)CCC1=O ; NCCOCCOCCN ; O=C(CCN1C(=O)C=CC1=O)NCCOCCOCCNC(=O)CCN1C(=O)C=CC1=O]

A 250 ml Schlenk flask was charged with maleimidopropionic acid-N-hydroxysuccinimide ester (ABCR, Karlsruhe, Germany, 1.24 g; 4.66 mmol) and dry dichloromethane (100 ml). To the resulting suspension triethylamine (650 μl; 4.66 mmol) was added followed by 2,2′-(ethylenedioxy)bis(ethylamine) (341 μl; 2.33 mmol) at room temperature. The resulting solution was stirred for 24 h. It was washed with 25 ml of a saturated sodium bicarbonate solution followed by 25 ml of brine. The organic phase was dried over sodium sulfate, filtered and evaporated to yield L6 (600 mg; 1.33 mmol; 57.1%) as a pink powder.

### Example E5

### Synthesis of Linker (L13)

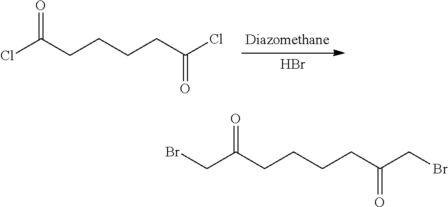 [SMILES: O=C(Cl)CCCCC(=O)Cl ; Br ; O=C(CBr)CCCCC(=O)CBr]

A solution of diazomethane in diethyl ether (0.245 mol/1) was prepared from DIAZALD® as described in T. H. Black, Aldrichimica Acta, 1983, 16, 3-10.

A 1 l one-neck flask was equipped with a magnetic stirring bar. An outside cooling (ice/water) was prepared. The flask was loaded with the diazomethane solution (500 ml). A dropping funnel with pressure exchange was installed. A solution of freshly distilled hexanedioyl-dichloride (4.58 g) in 30 ml diethyl ether was added drop-wise under slight development of gas. The funnel was washed with 20 ml diethyl ether and the mixture was stirred for 1 h at 0° C. An aqueous HBr-solution (9.5 ml, 62% (w/w)) was poured to this reaction mixture in one portion under a strong development of gas. A precipitate appeared. The temperature was allowed to warm to room temperature and the resulting mixture was stirred overnight. Diethyl ether was added (6 l) to dissolve the precipitate. The resulting solution was washed three times with 200 ml of water. The organic phase was dried with sodium sulfate and filtered. Activated charcoal (2.4 g) was added; the mixture was stirred for 30 min and filtered over kieselguhr. Again the organic phase was dried with sodium sulfate and filtered. Then 80-90% of the solvent were evaporated under reduced pressure at room temperature. The precipitate was collected by filtration, washed with cold diethyl ether and dried in vacuo. L13 was obtained in 47% yield (3.5 g) and used without further purification.

### Example E6

### Synthesis of Linker (L14)

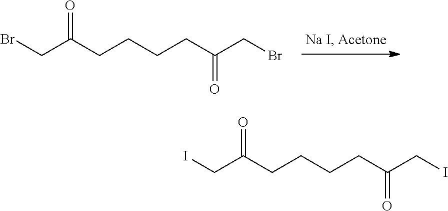

A 25 ml two-neck flask was equipped with a magnetic stirring bar under inert atmosphere. The flask was loaded with sodium iodide (1.1 g), evacuated and refilled with nitrogen. Under inert atmosphere acetone (7 ml) was added and the mixture was stirred until the salt dissolved. In a nitrogen flow L13 (1 g) was added and the resulting mixture was stirred at room temperature for 3 h. Then a further portion of sodium iodide (0.1 g) was added. The reaction was monitored by GC.

The solvent was removed at room temperature under reduced pressure and dichloromethane (100 ml) was added. The residual solid was filtrated and washed with dichloromethane (5 ml). The combined organic phases were washed with 20 ml of an aqueous solution of sodium hydrogensulfite/sodium sulfite and twice with 10 ml of water, dried with sodium sulfate and filtered. Approximately 75% of the solvent were evaporated under reduced pressure at room temperature. The precipitate was collected by filtration and dried in vacuo. L14 was obtained in 81% yield (1.06 g) as a white to off-white solid.

### II: Synthesis of Thiol Modified Hydroxyethyl Starch (Thiol-Modified HES)

### General Procedure E7

### Preparation of Thiol-Modified HES

10 g HES (M_{w} between 10 kDa and 700 kDa) were dissolved in 23 ml sodium acetate buffer (pH 5.0 and c=1 mol/1) by vigorous stirring and heating (up to 60° C.). To the clear solution, the crosslinking compound (II) (concentration varied from 0.18 to 2 mol/l, see Table 2) and NaCNBH₃ (concentration according to Table 2) were added. The reaction mixture was stirred at 60° C. for 16 to 24 h, diluted with water to 100 ml, neutralized with diluted sodium hydroxide solution, purified by ultrafiltration with a membrane (e.g. MWCO 10 kDa) against 2 l ultrapure water and concentrated to approximately 100 ml. 1 g sodium borohydride was added and the reaction mixture was stirred at 25° C. for 18 h. In case of cysteamine as crosslinking compound (II), the reaction mixture was stirred for 2 h. The pH was adjusted to 4.0 with 1M aqueous HCl solution and the thiol-modified HES was purified by ultrafiltration (e.g. membrane MWCO 10 kDa) against 1.5 l of a 10 mmol/l sodium acetate buffer pH 4.0, containing 1 mmol/l EDTA and subsequently with 500 ml ultrapure water and lyophilized. The RGC of thiol-modified HES was determined with Ellman's Reagent as described in Instructions Ellman's Reagent, Pierce Biotechnology, Inc. 7/2004, USA.

For determination of M_{w} and Mₙ see Example E10.

### III: Synthesis of Hydroxyethyl Starch Linker Derivatives

On the one hand, for a given linker structure, thiol-modified HES was varied with respect to the mean molecular weight, and with respect to its molar substitution. On the other hand, the chemical nature of the linker was varied, for a given thiol-modified HES starting material. The reaction details are summarized in Table 3.

### Example E8

### General Synthesis Procedure

The amounts A1 of thiol-modified HES X (weight average molecular weight Mw and molecular substitution MS see Table 2) were dissolved in the appropriate volume V1 of solvent S1 by stirring at room temperature. To the clear solution, the indicated amount A2 of linker L was added dissolved in a given volume V2 of solvent S2. The reaction mixture was incubated for time t at 21° C. at a mixing rate of 750 rpm (see Table 3). All experiments with iodine containing linkers were performed in the dark.

Work-Up Procedure 1 (for Samples with <1 g Thiol-Modified HES)

For work-up the solution was poured into seven-fold excess volumes of 2-propanol and centrifuged at room temperature for 15 min at 7000*g. The supernatant was discarded and the precipitate dissolved in ultrapure water to a final concentration of 5% (w/v).

The product was purified by size exclusion chromatography using ultrapure water as eluent, a guard column HiTrap™ Desalting 1*5 ml and a separation column HiPrep™ 26/10 Desalting 53 ml (both GE Healthcare). In each run 5 ml samples were injected. The chromatographic procedure was monitored by UV spectroscopy at the wavelength of 210 nm and 5 ml-fractions were collected. After use the column was equilibrated with 5 column volumes of 0.5 M acetic acid and 5 column volumes of ultrapure water. The fraction containing the HES-derivative were pooled and lyophilized.

Work-Up Procedure 2 (for Samples with ≧1 g Thiol-Modified HES)

All samples with more than 1 g thiol-modified HES were diluted to a final concentration of maximum 10% (v/v) DMF or DMSO and filtered. The HES-linker derivative was purified by ultrafiltration with a membrane (e.g. MWCO 10 kDa) against 20 times its own volume of ultrapure water and lyophilized.

### IV: Analysis of the Hydroxyethyl Starch Linker Derivatives

### Example E9

### Determination of Reactive Group Content of HES-Linker Derivatives

### General Synthesis Procedure

Between 4 and 25 mg HES-linker derivative was dissolved either in 0.21M phosphate buffer pH 8.5 containing 5 mM EDTA (linker L10-L14) or PBS-buffer pH 6.5 containing 5 mM EDTA (linker L1-L6). One equivalent of thiol T (referred to Mₙ of the HES species; see Table 3) was dissolved in 5 mM EDTA solution pH 6.0 and added to the HES-linker derivative solution. The final concentration of HES-linker derivative in the reaction mixture was 15% (w/v). The reaction mixture was incubated at 21° C. for 2 h. The samples were analyzed by RP-HPLC/UV (conditions see below in Example E11c) at the UV maximum of T (for peptides 280 nm and for PET 254 nm). The RGC was evaluated by comparison of the relative peak area of the conjugate to the sum of all other products. The reaction conditions for the various target molecules were not optimized.

### Example E10

### General Procedure for the Determination of the Mean Molecular Weight M_{w}

M_{w} and Mₙ were determined as described in WO 2012/004007 A1, Example 1.9.

### Example E11

### Stability Studies

### a) General Synthesis Procedure: Synthesis of HES-Linker-(2-Mercaptoethyl)Pyrazine Conjugates

Between 150 and 200 mg HES-linker derivative (defined by the linker and the HES species) was dissolved in 0.1M borate buffer pH 8 containing 5 mM EDTA to a concentration of 20% (w/v). (2-Mercaptoethyl)pyrazine (20 equivalents referred to Mₙ of the HES species) was dissolved in the same volume of DMF and added to the HES-linker derivative solution. The reaction mixture was incubated at 21° C. for 16-24 h. After incubation the samples were purified by first precipitation and subsequently desalting and isolated by freeze drying as described above in example E8. The conjugates were analyzed by RP-HPLC (for conditions see below). The reaction conditions for the various target molecules were not optimized.

### b) Evaluation of the Stability of HES-Linker-(2-Mercaptoethyl)Pyrazine Conjugates in Aqueous Buffer Solutions of Different pH

The conjugates were dissolved in buffer and diluted to a concentration of 20 mg/ml. The stability study was performed in a) 0.1M sodium acetate buffer pH=4.0, b) 0.1M sodium phosphate buffer pH=7.0 or c) 0.1M sodium borate buffer pH=8.0. The samples were incubated for up to 20 d at 40° C. Samples were taken after 0, 1, 5, 10 and 20 d. They were analyzed by RP-HPLC/UV at 266 nm (for conditions see below). The decay was evaluated by comparison of the relative peak area of the conjugate to the sum of all decomposition products.

### c) RP-HPLC Analysis of HES-Linker-(2-Mercaptoethyl)Pyrazine Conjugates

The product was analyzed by RP-HPLC using ultrapure water with 0.1% TFA (Uvasol®, for spectroscopy, Merck, Code No. 1.08262.0100) as eluent A and acetonitrile (Uvasol®, Reag. Ph. Eur., gradient grade for liquid chromatography, Code No. 1.00030.2500) with 0.1% TFA as eluent B. The analysis was performed with a pre-column SecurityGuard™ Cartridge system, Widepore C18, ODS, 4 mm L*3.0 mm ID (Phenomenex, Code No. AJO-4321) and a separation column Reprosil Gold 300, C18, 5μ, 150*4.6 mm (Dr. Maisch, Code No. r35.9g.s1546, SN: 120109 251501). In each run 100 μl samples were injected. The samples were analyzed with the following gradient with a flow of 2 ml/min:

| | | | |
|---|---|---|---|
| 0-2.5 | min | 2% | B |
| 2.5-10 | min | 2-60% | B |
| 10-12.6 | min | 98% | B |
| 12.7-15.3 | min | 2% | B |

### V. Conjugation of the HES Derivatives to Proteins

### Example C1

### Conjugation of Thiol-Reactive HES and Ubiquitin

Ubiquitin (Ubi, pdb code: 1UBQ) was selected as model protein for testing reactivity and stability of various thiol-reactive HES derivatives. For this purpose the protein variant Ubi F45W S57C (with a C-terminal His6 tag, manufactured by Scil Proteins, Halle, Germany) was used allowing site-specific conjugation to the single cysteine residue introduced on position 57 and detection by UV spectroscopy by the tryptophan residue introduced on position 45.

To avoid dimerization of the protein and to get a high yield of conjugate, Ubi had to be reduced with DTT before starting the coupling procedure. DTT is used in a 50fold molar excess and the reduction takes place for 1 hour at 37° C. Afterwards the DTT was removed by cation exchange procedure on a 1 ml HiTrap SP HP (GE Healthcare). DTT elutes from the column in the flow-through, afterwards Ubi was eluted by a step gradient.

For small-scale conjugation reactions, a 10% or 40% (w/v) stock solution of the thiol-reactive HES derivative (defined by the linker and the HES species) was prepared. The appropriate amount of HES derivative was weighed into the reaction tubes and dissolved in reaction buffer until a clear solution appeared. The protein solution and the HES derivatives were combined in a specified ratio (Table 4) and mixed thoroughly. For large scale conjugation reactions, the appropriate amount of thiol-reactive HES derivative (see Table 4) was weighed directly in a 15 ml Falcon tube, dissolved in reaction buffer (see Table 4) as described above and mixed thoroughly with the appropriate protein solution. The reaction mixtures were analyzed by either SEC (example see FIG. 2), RP-HPLC and SDS-PAGE. The chromatogram monitored at a wavelength of 220 nm or 280 nm was integrated and the yield of the conjugation reaction was calculated from the peak areas of the conjugate and the non-modified protein. The coupling procedure was optimized for different pH conditions and various HES:target ratios (Table 4).

The preparation of the HES-Ubi conjugates was performed by cation exchange chromatography. All chromatographic steps were performed at room temperature using an Akta Purifier 100 system (GE Healthcare) and monitored by UV spectroscopy at a wavelength of 220 nm and 280 nm and by conductivity measurements. For the preparation of the HES-Ubi conjugates a HiTrap SP HP 1 ml column (GE Healthcare) was used. Eluents were exchanged to eluent A (20 mM acetate, pH 4.0) and eluent B (20 mM acetate, pH 4.0, 1 M NaCl); the column was equilibrated with 10 CV eluent A. The reaction mixture was diluted approximately 20fold using eluent A and loaded onto the column using the sample pump. The flow-through was collected in 50 ml Falcon tubes. Unbound sample was washed out with five CV eluent A and the conjugate was eluted with a flow rate of 1.5 ml/min and a segmented salt gradient (FIG. 3). Fractions containing HES-Ubi conjugates free of unmodified Ubi were combined and concentrated in a suitable centrifugal concentrator (e.g. Amicon Ultra 4, 10 kDa MWCO) and sterile-filtered using a 0.22 μm syringe filter with low protein binding (Pall Acrodisc). The concentration was determined by UV spectroscopy. Stability studies were conducted as described in example C6.

### Example C2

### Conjugation of Thiol-Reactive HES and IL1RA (Kineret®)

Coupling reactions of IL1RA (pdb code: 1IRA) with HES derivatives were performed as described in example C1 (examples listed in Table 4). The reaction mixtures were analyzed by RP-HPLC (as described in example C1) with a Jupiter C18 column (300 Å, 5 μm, 4.6×150 mm, Phenomenex) with a segmented gradient (0-0.2 min: 2% B, 0.2-0.8 min: 2-30% B, 0.8-5.8 min: 30-40% B, 5.8-6.5 min: 98% B, 6.5-7.0 min: 98% B, 7.0-9.0 min: 2% B) and a flow rate of 1 ml/min or by SEC (as described in example C1) with a flow rate of 0.5 ml/min within 60 min on a Superose 6 10/300 GL (GE Healthcare) column.

Preparations of conjugates for stability tests were performed in a similar way as described in example C1 by anion exchange chromatography with two HiTrap Q HP 1 ml columns (GE Healthcare) under following conditions: flow rate 1 ml/min, eluent A: 10 mM Tris/HCl, pH 8.0, eluent B: 10 mM Tris/HCl, 0.25 M NaCl, pH 8.0, loaded sample 5fold diluted and a segmented gradient (5 CV: 0% B, 6 CV: 25% B, 11 CV: 25-36% B, 5 CV: 100% B, 5 CV: 0% B). Fractions containing the protein conjugate were combined and concentrated in a centrifugal concentrator. Stability studies were conducted as described in example C6.

Conjugates of L12-HES with IL1RA were tested for binding affinity to its natural binding partner using SPR on a BIAcore system. Interleukin 1 receptor type I (R&D Systems) was immobilized on the chip surface and the kinetic binding parameters were determined for the conjugate in comparison to the unmodified protein (chip: CM3, immobilization: 950 RU, flow rate 30 μl/min, association 180 s, dissociation 900 s, regeneration: 6 s with 5 mM glycine buffer, pH 2.0). The curves were analyzed by assuming a 1:1 binding stoichiometry. The L12-HES-IL1RA conjugate retained an excellent binding affinity with a K_{D} value of 204 pM in comparison to 90 pM for the unmodified IL1RA and 187 pM for IL1RA that is conjugated to HES via N-terminal coupling. The conjugate shows an association rate kₐ of 2.44·10⁵ M⁻¹s⁻¹ that is comparable to unmodified IL1RA (kₐ=5.88·10⁵ M⁻¹s⁻¹) and IL1RA that is conjugated to HES via N-terminal coupling (kₐ=2.63·10⁵ M⁻¹s⁻¹).

### Example C3

### Conjugation of Thiol-Reactive HES and A1AT

A1AT (pdb code: 1KCT) contains a single cysteine that is partially capped and therefore had to be reduced before starting the coupling procedure by addition of a 10fold molar excess of DTT for one hour at 37° C. After reduction, the DTT was removed by buffer exchange in a centrifugal concentrator.

Coupling reactions of A1AT with HES derivatives were performed as described in example C1 (examples listed in Table 4). The reaction mixtures were analyzed by RP-HPLC with a Jupiter C18 column (300 Å, 5 μm, 4.6×150 mm) as described in example C1 with a segmented gradient (0-4 min: 5% B, 4-8 min: 5-46% B, 8-17 min: 46-51% B, 17-22 min: 98% B, 22-25 min: 5% B) and a flow rate of 1 ml/min.

The activity of the L12-HES-A1AT conjugate was analyzed in an elastase inhibition assay as described in Beatty et al. (1980, JBC, 255, 9, 3931-3934) and compared to unmodified A1AT. The L12-HES-A1AT conjugate shows 95% of the elastase inhibition activity compared to unmodified A1AT.

### Example C4

### Conjugation of Thiol-Reactive HES and SlyD

SlyD D101C (Zoldak and Schmid, 2011, JMB, 406(1), 176-94; pdb code: 2K8I) contains a single additional cysteine that had to be reduced by addition of a 10fold molar excess of DTT for one hour at 37° C. After reduction, the DTT was removed by buffer exchange in a centrifugal concentrator.

A coupling reaction of SlyD with a HES derivative was performed as described in example C1 (see Table 4). The reaction mixture was analyzed by RP-HPLC with a Jupiter C18 column (300 Å, 5 μm, 4.6×150 mm) as described in example C1 with a segmented gradient (0-1 min: 2-30% B, 1-6 min: 30-50% B, 6-10 min: 98% B, 10-13 min: 2% B) and a flow rate of 2 ml/min or by SEC (as described in example C1) with a flow rate of 0.5 ml/min within 60 min on a Superose 12 10/300 GL (GE Healthcare) column.

Preparations of conjugates for stability tests were performed in a similar way as described in example C1 by anion exchange chromatography with a Q Ceramic HyperD F1 ml column (PALL) and the following conditions: flow rate 1 ml/min, eluent A: 20 mM Tris/HCl, pH 8.0, eluent B: 20 mM Tris/HCl, 1 M NaCl, pH 8.0, loaded sample 10fold diluted and a segmented gradient (5 CV: 0% B, 20 CV: 0-40% B, 5 CV: 100% B, 5 CV: 0% B). Fractions containing the protein conjugate were combined and concentrated in a centrifugal concentrator. Stability studies were conducted as described in example C6.

Prolylisomerases like SlyD catalyze the trans to cis isomerization of peptidyl prolyl bonds. The prolyl isomerase activities of SlyD and the L12-HES-SlyD conjugate were measured by a prolyl isomerase activity assay according to Zoldak et al. (2009, Biochemistry, 48, 10423-10436). The rate constant of the cis to trans isomerization of the prolyl bond was determined using Origin 8.1. The L12-HES-SlyD conjugate shows an equal activity for prolyl isomerization as unmodified SlyD. The catalytic activity of unmodified SlyD is 5.6·10⁶ M⁻¹s⁻¹ and the activity of the L12-HES-SlyD conjugate is 6.0·10⁶ M⁻¹s⁻¹ (107% of the activity of unconjugated SlyD). The higher activity of the conjugate compared to unmodified SlyD is within the error of the assay.

### Example C5

### Conjugation of Thiol-Reactive HES and HSA

Coupling reactions of HSA (pdb code: 1E7H) with HES derivatives were performed as described in example C1, examples are shown in Table 4. The reaction mixtures were analyzed by SEC (as described in example C1) with a Superose 6 10/300 GL column with a flow rate of 0.5 ml/min within 60 min as described in example C1.

Preparations of conjugates for stability tests were performed in a similar way as described in example C1 by anion exchange chromatography with a HiTrap Q HP 5 ml column under following conditions: flow rate 1 ml/min, eluent A: 20 mM Tris/HCl, pH 8.0, eluent B: 20 mM Tris/HCl, 1 M NaCl, pH 8.0, loaded sample 10fold diluted and a segmented gradient (5 CV: 0% B, 40 CV: 0-30% B, 5 CV: 100% B, 5 CV: 0% B). Fractions containing the protein conjugate were combined and concentrated in a centrifugal concentrator. Stability studies were conducted as described in example C6.

### Example C6

### General Description for Coupling Procedure

The amount of the target molecule indicated in Table 4 was transferred into the appropriate reaction buffer. The indicated amount of HES derivative (defined by the linker and the HES species) was dissolved in reaction buffer and mixed with the target substance solution. The HES species used for the conjugation to HSA had an approximate molecular weight of 250 kDa, for all other targets a HES species with a molecular weight between 60 and 90 kDa was used (see Table 2). HES:target describes the molar ratio of reactive groups of HES to protein concentration (target). The coupling reactions were performed in 0.1 M phosphate with 5 mM EDTA or 0.1 M Tris/HCl depending on the required pH for two hours at 20 or 25° C. (Ubi and A1AT) or overnight at 5° C. (all other targets).

### Stability Tests

For stability tests at pH 4.0, 5.5, 7.0 and 8.0, the conjugates were diluted into final buffer conditions of 20 mM acetate (pH 4.0 and 5.5) or 10 mM phosphate (pH 7.0 and 8.0), 0.5 mM ETDA, 154 mM NaCl to a final concentration of 1 mg/ml. The conjugates were stored at 40° C. for 20 days and analyzed by RP-HPLC (example for Ubi is shown in FIG. 3), SEC (as described in example C1) or SDS-PAGE (as described in example C1). The results obtained with different target molecules are given in Table 5 and in table 6 and FIGS. 4-6.

As may be taken from table 5 and table 6, linker L12 shows a surprisingly high stability, in particular at physiologically pH or lower pH ranges.

### VI: Specificity of Derivatization

### Example E12

### Synthesis of L12-HES10/1.0 to Show Specificity of the Reaction Between HES and Linker

A HES derivative with the linker L12 was synthesized from a thiol-modified HES species 10/1.0 (according to example E7 and Table 2 (entry 11)) following the procedure of example E8 (table 3, entries 21). In parallel a control reaction was conducted under comparable conditions with an underivatized HES (table 3, entries 22). Both samples were subjected to the identical purification process and the content of thiol-reactive groups assessed using the PET assay described in example E9.

The analysis of the reactive group content shown in Table 3, entry 21 and entry 22, shows that under the reaction conditions according to the invention no side reactivity of HES with the linker L12 was observed.

### VII. Unspecific Derivatization with Linker Compounds Comprising Activated Carboxy Groups (Example not According to the Invention)

### Example E13

### Treatment of HES with 3-(4-Hydroxyphenyl)Propionic Acid N-Hydroxysuccinimide Ester to Demonstrate that HES Readily Reacts with Activated Carboxy Groups (Example not According to the Invention)

53.8 mg HES 100/1.0/1.4 (Mn=59 kDa, Mw=80 kDa) were dissolved in 192 μl PBS (25 mM Na phosphate, 100 mM NaCl pH 7.5), 10 mg of 3-(4-Hydroxyphenyl)propionic acid N-hydroxysuccinimide ester (Sigma, Germany) were dissolved in 100 μl DMSO. 5 μl of HES solution was mixed with the specified amount of 3-(4-Hydroxyphenyl)propionic acid N-hydroxysuccinimide ester and PBS and incubated for over night at 4° C.

| HES: | A | | B | | C | | D | |
|---|---|---|---|---|---|---|---|---|
| 3-(4-Hydroxyphenyl)pro- | 1:4 | | 1:8 | | 1:20 | | 1:40 | |
| pionic acid N-hydroxy- | | | | | | | | |
| succinimide ester | | | | | | | | |
| HES | 5 | μl | 5 | μl | 5 | μl | 5 | μl |
| 3-(4-Hydroxyphenyl)pro- | 0.904 | μl | 1.808 | μl | — | | — | |
| pionic acid N-hydroxy- | | | | | | | | |
| succinimide ester 20 g/l | | | | | | | | |
| 3-(4-Hydroxyphenyl)pro- | — | | — | | 0.904 | μl | 1.808 | μl |
| pionic acid N-hydroxy- | | | | | | | | |
| succinimide ester 100 g/l | | | | | | | | |
| PBS | 94.1 | μl | 93.2 | μl | 94.1 | μl | 93.2 | μl |

Samples were analysed by size exclusion chromatography using a BioSep-SEC-S 3000 300×7.8 mm, 5 μm (Phenomenex, Germany) at a flow rate of 1 ml/min and 50 mM Na-phosphate, pH 8.0, 300 mM NaCl as running buffer.

Analysis by size exclusion chromatography reveals that when HES is contacted with a compound comprising an activated carboxy group, unmodified HES reacts with said compound, in this case with 3-(4-Hydroxyphenyl)propionic acid N-hydroxysuccinimide ester, to give a modified HES (see FIG. 7).

TABLE 1
Homo-bifunctional linker structures

| | | molecular | | | |
|---|---|---|---|---|---|
| | | weight | | product | |
| entry | linker | [g/mol] | vendor | no. | linker structure |
| 1 | L1 | 400.61 | synthesis described in example E1 | — | 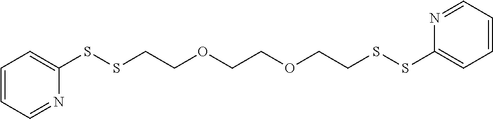 [SMILES: c1ccc(SSCCOCCOCCSSc2ccccn2)nc1] |
| 2 | L2 | 482.70 | Pierce | 21702 | 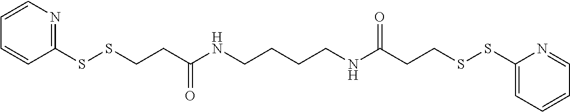 [SMILES: O=C(CCSSc1ccccn1)NCCCCNC(=O)CCSSc1ccccn1] |
| 3 | L3 | 308.29 | Pierce | 22336 | 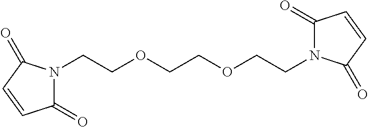 [SMILES: O=C1C=CC(=O)N1CCOCCOCCN1C(=O)C=CC1=O] |
| 4 | L4 | 220.18 | Pierce | 22323 | 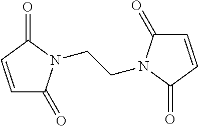 [SMILES: O=C1C=CC(=O)N1CCN1C(=O)C=CC1=O] |
| 5 | L5 | 248.23 | Pierce | 22331 | 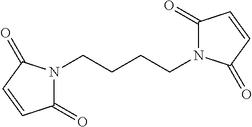 [SMILES: O=C1C=CC(=O)N1CCCCN1C(=O)C=CC1=O] |
| 6 | L6 | 450 | synthesis described in example E2 | — | 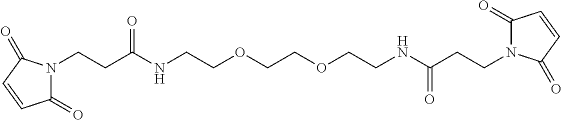 [SMILES: O=C(CCN1C(=O)C=CC1=O)NCCOCCOCCNC(=O)CCN1C(=O)C=CC1=O] |
| 7 | L10 | 266.38 | Uptima | UPTIUPL 7733A | 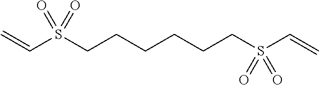 [SMILES: C=CS(=O)(=O)CCCCCCS(=O)(=O)C=C] |
| 8 | L11 | 324.37 | TCI | B1746 | 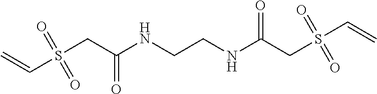 [SMILES: C=CS(=O)(=O)CC(=O)NCCNC(=O)CS(=O)(=O)C=C] |
| 9 | L12 | 118.15 | Sigma- Aldrich | V3700 | 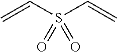 |
| 10 | L13 | 299.99 | synthesis described in example E5 | — | 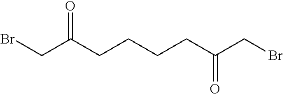 [SMILES: O=C(CBr)CCCCC(=O)CBr] |
| 11 | L14 | 393.99 | synthesis described in example E6 | — | 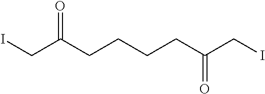 [SMILES: O=C(CI)CCCCC(=O)CI] |

TABLE 2
Thiol-modified HES species (example E7)

| | | | | crosslinking | concentration of | concentration of | | |
|---|---|---|---|---|---|---|---|---|
| | | | thiol-modified | compound | crosslinking compound | NaBH₃CN | | |
| entry | MS | M_{W} [kDa] | HES X | (II) | (II) [mol/l] | [mol/l] | RGC [%] | M_{w} [kDa] |
| 1 | 1.0 | 83 | X2 | cystamine * 2HCl | 0.6 | 0.6 | 65 | 84 |
| 2 | 1.3 | 84 | X6 | cystamine * 2HCl | 0.18 | 0.6 | 54 | 84 |
| 3 | 1.0 | 78 | X7 | cystamine * 2HCl | 1.433 | 0.6 | 70 | 84 |
| 4 | 1.3 | 84 | X8 | cystamine * 2HCl | 0.18 | 0.6 | 53 | 84 |
| 5 | 1.3 | 84 | X9 | cystamine * 2HCl | 0.18 | 0.6 | 50 | 85 |
| 6 | 1.0 | 80 | X11 | cystamine * 2HCl | 0.6 | 0.6 | 61 | 80 |
| 7 | 1.0 | 91 | X12 | cysteamine * HCl | 1.0 | 0.3 | 83 | 92 |
| 8 | 1.0 | 29 | X18 | cysteamine * HCl | 2.0 | 0.6 | 80 | 29 |
| 9 | 1.0 | 247 | X19 | cysteamine * HCl | 2.0 | 0.6 | 74 | 246 |
| 10 | 1.0 | 86 | X20 | cysteamine * HCl | 2.0 | 0.6 | 76 | 85 |
| 11 | 1.0 | 10.9 | X1 | cystamine * 2HCl | 1.43 | 0.6 | 60 | 11.2 |

TABLE 3
Variation of the linker structure (example E8)

| | | | | derivatization | | | | | | | | | | | |
|---|---|---|---|---|---|---|---|---|---|---|---|---|---|---|---|
| starting material | | | | | | E: | | V1: volume | | V2: volume | | | | | |
| | linker | thiol-modified | | A1: thiol-modified | A2: linker | equivalents | | solvent | | solvent | | work-up | | | |
| entry | L # | HES species X | MS | HES amount [mg] | amount [mg] | [eq] | S1: solvent | S1 [ml] | S2: solvent | S2 [ml] | t: time [h] | procedure | M_{w} [kDa] | RGC [%] | thiol T |
| 1 | L1 | X6 | 1.3 | 100.41 | 7.90 | 20 | PBS | 0.8 | DMF | 0.2 | 1 | 1 | n.d. | 36 | R6R |
| | | | | | | | pH 6.5* | | | | | | | | |
| 2 | L1 | X18 | 1.0 | 1000 | 137 | 10 | PBS | 8 | DMF | 2 | 1 | 2 | 35 | 36 | Ac—R6R—NH2 |
| | | | | | | | pH 6.5* | | | | | | | | |
| 3 | L1 | X19 | 1.0 | 1000 | 15 | 10 | PBS | 8 | DMF | 2 | 1 | 2 | 251 | 48 | Ac—R6R—NH2 |
| | | | | | | | pH 6.5* | | | | | | | | |
| 4 | L1 | X20 | 1.0 | 1000 | 49 | 10 | PBS | 8 | DMF | 2 | 1 | 2 | 99 | 49 | Ac—R6R—NH2 |
| | | | | | | | pH 6.5* | | | | | | | | |
| 5 | L2 | X7 | 1.0 | 1001.32 | 29.35 | 5 | PBS | 8 | DMF | 2 | 1 | 2 | 83 | 40 | Ac—R6R—NH₂ |
| | | | | | | | pH 6.5* | | | | | | | | |
| 6 | L3 | X8 | 1.3 | 500.64 | 7.46 | 5 | PBS | 5 | DMF | 1 | 1 | 1 | n.d. | 40 | R6R |
| | | | | | | | pH 6.5* | | | | | | | | |
| 7 | L4 | X9 | 1.3 | 1002.28 | 50.22 | 25 | PBS | 8 | DMF | 2 | 1 | 2 | 89 | 53 | Ac—R6R—NH₂ |
| | | | | | | | pH 6.5* | | | | | | | | |
| 8 | L5 | X6 | 1.3 | 501.27 | 24.47 | 20 | PBS | 4 | TFE | 1 | 1 | 1 | n.d. | 29 | R6R |
| | | | | | | | pH 6.5* | | | | | | | | |
| 9 | L6 | X9 | 1.3 | 502.77 | 41.02 | 20 | PBS | 4 | PBS | 1 | 1 | 1 | 91 | 43 | Ac—R6R—NH₂ |
| | | | | | | | pH 6.5* | | pH 6.5* | | | | | | |
| 10 | L10 | X7 | 1.0 | 1008.11 | 27.40 | 10 | Borate | 8 | DMF | 2 | 1 | 2 | 81 | 63 | Ac—R6R—NH₂ |
| | | | | | | | pH 8** | | | | | | | | |
| 11 | L10 | 18 | 1.0 | 1000 | 45 | 5 | Borate | 8 | DMF | 2 | 1 | 2 | 35 | 59 | Ac—R6R—NH2 |
| | | | | | | | pH 8** | | | | | | | | |
| 12 | L10 | X19 | 1.0 | 1000 | 10 | 10 | Borate | 8 | DMF | 2 | 1 | 2 | 251 | 69 | Ac—R6R—NH2 |
| | | | | | | | pH 8** | | | | | | | | |
| 13 | L10 | X20 | 1.0 | 1000 | 32 | 10 | Borate | 8 | DMF | 2 | 1 | 2 | 95 | 74 | Ac—R6R—NH2 |
| | | | | | | | pH 8** | | | | | | | | |
| 14 | L11 | X11 | 1.0 | 2025.28 | 157.28 | 25 | DMSO | 20 | — | — | 18 | 2 | 83 | 48 | Ac—R6R—NH₂ |
| 15 | L12 | X12 | 1.0 | 1000 | 7.6 | 5 | DMSO/buffer | 4 | — | — | 1 | 2 | 94 | 83 | Ac—R6R—NH₂ |
| | | | | | | | 8:2*** | | | | | | | | |
| 16 | L12 | X18 | 1.0 | 1000 | 40.3 | 10 | DMSO/buffer | 10 | — | — | 1 | 2 | 30 | 85 | Ac—R6R—NH2 |
| | | | | | | | 8:2*** | | | | | | | | |
| 17 | L12 | X19 | 1.0 | 1000 | 4 | 10 | DMSO/buffer | 10 | — | — | 1 | 2 | 247 | 85 | Ac—R6R—NH2 |
| | | | | | | | 8:2*** | | | | | | | | |
| 18 | L12 | X20 | 1.0 | 1000 | 14 | 10 | DMSO/buffer | 10 | — | — | 1 | 2 | 93 | 82 | Ac—R6R—NH2 |
| | | | | | | | 8:2*** | | | | | | | | |
| 19 | L13 | X2 | 1.0 | 15000 | 3750 | 85 | DMF | 150 | — | — | 17.5 | 2 | 88 | 48 | Ac—R6R—NH₂ |
| 20 | L14 | X9 | 1.0 | 1003.14 | 250 | 70 | DMF | 10 | — | — | 18 | 2 | 87 | 39 | Ac—R6R—NH₂ |
| 21 | L12 | X1 | 1.0 | 502 | 42.6 | 10 | acetate | 1 | DMSO | 4 | 1 | 1 | 11.6 | 50.1 | PET |
| | | | | | | | pH 4.0 | | | | | | | | |
| 22 | L12 | HES**** | 1.0 | 212 | 32.5 | 10 | acetate | 0.5 | DMSO | 2 | 1 | 1 | 8.7 | 0 | PET |
| | | | | | | | pH 4.0 | | | | | | | | |

*PBS-buffer (25 mmol/l sodium phosphate, 135 mmol/l NaCl), 5 mmol/l EDTA, pH 6.5
**0.1 mol/l sodium borate buffer, 5 mmol/l EDTA, pH 8
***0.1 mol/l acetate buffer + 5 mmol/l EDTA, pH 4
****HES not modified with SH, example not according to the invention

TABLE 4
Examples of coupling reactions: variation of linker molecules, target substances,
buffer conditions and HES:target ratios

| | Thiol- | | | | | | | |
|---|---|---|---|---|---|---|---|---|
| | modified | | | | HES | target | HES | yield |
| Linker L | HES species X | target | pH | HES:target | [% w/v] | [mg] | [mg] | [%] |
| L1 | X18 | Ubi | 7.0 | 1:1 | 1.5 | 2.5 | 25.4 | 94 |
| L1 | X19 | Ubi | 7.0 | 1:1 | 6.0 | 2.5 | 105.3 | 92 |
| L1 | X20 | Ubi | 7.0 | 1:1 | 2.5 | 2.5 | 40.5 | 96 |
| L3 | X8 | Ubi | 7.0 | 5:1 | 13.9 | 0.05 | 3.83 | 41 |
| L5 | X6 | Ubi | 7.0 | 5:1 | 15.1 | 0.05 | 4.37 | 43 |
| L10 | X7 | Ubi | 7.0 | 1.5:1 | 2.6 | 0.05 | 0.92 | 18 |
| L10 | X7 | Ubi | 7.5 | 1.5:1 | 2.6 | 0.05 | 0.92 | 27 |
| L10 | X7 | Ubi | 8.0 | 1.5:1 | 2.6 | 0.05 | 0.92 | 33 |
| L10 | X7 | Ubi | 8.5 | 1.5:1 | 2.6 | 0.05 | 0.92 | 30 |
| L10 | X7 | Ubi | 9.0 | 1.5:1 | 2.6 | 0.05 | 0.92 | 35 |
| L10 | X7 | Ubi | 8.5 | 1:1 | 1.9 | 0.05 | 0.61 | 24 |
| L10 | X7 | Ubi | 8.5 | 2:1 | 3.2 | 0.05 | 1.23 | 30 |
| L10 | X7 | Ubi | 8.5 | 3:1 | 4.1 | 0.05 | 1.84 | 33 |
| L10 | X18 | Ubi | 8.5 | 2:1 | 1.7 | 2.5 | 27.6 | 93 |
| L10 | X19 | Ubi | 8.5 | 2:1 | 7.9 | 2.5 | 147.6 | 94 |
| L10 | X20 | Ubi | 8.5 | 2:1 | 2.8 | 2.5 | 46.1 | 91 |
| L11 | X11 | Ubi | 7.0 | 1.5:1 | 7.3 | 0.05 | 1.40 | 39 |
| L11 | X11 | Ubi | 7.5 | 1.5:1 | 7.3 | 0.05 | 1.40 | 46 |
| L11 | X11 | Ubi | 8.0 | 1.5:1 | 7.3 | 0.05 | 1.40 | 34 |
| L11 | X11 | Ubi | 8.5 | 1.5:1 | 7.3 | 0.05 | 1.40 | 24 |
| L11 | X11 | Ubi | 8.5 | 3:1 | 17 | 0.05 | 4.21 | 81 |
| L12 | X12 | Ubi | 7.0 | 1.5:1 | 2.8 | 0.05 | 1.02 | 44 |
| L12 | X12 | Ubi | 7.5 | 1.5:1 | 2.8 | 0.05 | 1.02 | 78 |
| L12 | X12 | Ubi | 8.0 | 1.5:1 | 2.8 | 0.05 | 1.02 | 81 |
| L12 | X12 | Ubi | 8.5 | 1.5:1 | 2.8 | 0.05 | 1.02 | 78 |
| L12 | X12 | Ubi | 9.0 | 1.5:1 | 2.8 | 0.05 | 1.02 | 92 |
| L12 | X12 | Ubi | 8.5 | 1:1 | 2.0 | 0.05 | 0.68 | 65 |
| L12 | X12 | Ubi | 8.5 | 2:1 | 3.4 | 0.05 | 1.36 | 95 |
| L12 | X12 | Ubi | 8.5 | 3:1 | 4.4 | 0.05 | 2.04 | 100 |
| L12 | X12 | Ubi | 8.5 | 3:1 | 12.9 | 6.0 | 197.6 | 90 |
| L12 | X18 | Ubi | 8.5 | 2:1 | 0.9 | 2.5 | 14.6 | 95 |
| L12 | X19 | Ubi | 8.5 | 2:1 | 2.7 | 2.5 | 43.7 | 94 |
| L12 | X20 | Ubi | 8.5 | 2:1 | 6.7 | 2.5 | 120.8 | 96 |
| L13 | X2 | Ubi | 8.5 | 3:1 | 4.7 | 0.05 | 2.31 | 84 |
| L14 | X9 | Ubi | 8.5 | 3:1 | 5.2 | 0.05 | 2.79 | 81 |
| L12 | X12 | IL1RA | 8.5 | 5:1 | 6.6 | 10 | 328 | 51 |
| L1 | X19 | HSA | 7.0 | 3:1 | 15 | 5.0 | 92.2 | 88 |
| L10 | X19 | HSA | 8.5 | 10:1 | 15 | 5.0 | 215.2 | 31 |
| L12 | X19 | HSA | 8.5 | 10:1 | 15 | 5.0 | 176.1 | 61 |
| L12 | X12 | SlyD | 8.5 | 3:1 | 5.3 | 0.05 | 0.85 | 95 |
| L10 | X7 | A1AT | 8.0 | 5:1 | 3.7 | 0.05 | 0.53 | 35 |
| L11 | X11 | A1AT | 8.0 | 5:1 | 2.9 | 0.1 | 1.33 | 11 |
| L12 | X12 | A1AT | 8.0 | 5:1 | 2.5 | 0.1 | 1.10 | 39 |
| L12 | X12 | A1AT | 8.0 | 10:1 | 4.0 | 4.0 | 75.9 | 47 |

TABLE 5
Stability data of conjugates with various target proteins and
linker molecules

| | |
|---|---|
| Thiol- | |
| modified | conjugate degradation |
| HES | after 20 days (%) |

| Linker L | species X | target | pH 4.0 | pH 5.5 | pH 7.0 | pH 8.0 |
|---|---|---|---|---|---|---|
| L1 | X20 | Ubi | 0.4 | 0.2 | 4.3 | 10.8 |
| L2 | X7 | Ubi | 0.7 | n.d. | 24.1 | 29.0 |
| L4 | X9 | Ubi | 2.1 | n.d. | 15.2 | 17.4 |
| L10 | X20 | Ubi | 1.1 | 2.8 | 16.5 | 17.6 |
| L11 | X11 | Ubi | 2.2 | n.d. | 7.4 | 10.9 |
| L12 | X20 | Ubi | 0.3 | 0.0 | 0.8 | 3.0 |
| L13 | X2 | Ubi | 5.1 | n.d. | 5.5 | 11.4 |
| L14 | X9 | Ubi | 3.4 | n.d. | 5.8 | 10.6 |
| L10 | X7 | IL1RA | 6.0 | 7.9 | n.d. | 17.3 |
| L12 | X12 | IL1RA | 2.7 | 2.1 | 9.0 | 14.8 |
| L12 | X12 | SlyD | n.d. | 1.4 | 3.4 | 9.0 |
| L1 | X20 | HSA | 1.0 | 1.2 | 4.8 | 7.8 |
| L10 | X20 | HSA | 0.7 | 5.9 | 20.7 | 25.5 |
| L12 | X20 | HSA | 0.1 | 1.3 | 5.9 | 10.5 |
| L1 | X6 | MEP | 5.2 | n.d. | 24.3 | 25.2 |
| L2 | X7 | MEP | 1.4 | n.d. | 17.3 | 18.2 |
| L3 | X8 | MEP | 5.3 | n.d. | 6.8 | 7.8 |
| L4 | X9 | MEP | 2.5 | n.d. | 3.8 | 5.9 |
| L5 | X6 | MEP | 2.3 | n.d. | 10.5 | 12.5 |
| L6 | X9 | MEP | 10.7 | n.d. | 9.4 | 10.2 |
| L10 | X7 | MEP | 4.0 | n.d. | 15.4 | 15.9 |
| L11 | X11 | MEP | 5.8 | n.d. | 2.4 | 6.0 |
| L12 | X12 | MEP | 0.2 | n.d. | 0.0 | 0.8 |
| L13 | X2 | MEP | 2.5 | n.d. | 4.9 | 7.1 |

TABLE 6
Stability data of conjugates with Ubi or with MEP

| | | | conjugate degradation after 20 days | | | |
|---|---|---|---|---|---|---|
| | Thiol-modified | | (%) | | | |
| linker | HES species X | target | pH 4.0 | pH 5.5 | pH 7.0 | pH 8.0 |
| L1 | X18 | Ubi | 0.5 | 0.4 | 15.4 | 23.4 |
| L10 | X18 | Ubi | 0.8 | 2.0 | 9.6 | 10.8 |
| L12 | X18 | Ubi | 0.5 | 0.5 | 1.0 | 2.2 |
| L1 | X19 | Ubi | 0.3 | 0.5 | 7.1 | 15.7 |
| L10 | X19 | Ubi | 1.6 | 3.1 | 17.4 | 19.2 |
| L12 | X19 | Ubi | 0.4 | 0.6 | 1.0 | 2.8 |
| L1 | X20 | Ubi | 0.4 | 0.2 | 4.3 | 10.8 |
| L10 | X20 | Ubi | 1.1 | 2.8 | 16.5 | 17.6 |
| L12 | X20 | Ubi | 0.3 | 0.0 | 0.8 | 3.0 |
| | Thiol-modified | | conjugate degradation after 20 days | | | |
| linker | HES species X | target | (%)* | | | |
| L1 | X18 | MEP | 13.9 | n.d. | 17.5 | 17.8 |
| L10 | X18 | MEP | 0.9 | n.d. | 8.0 | 7.2 |
| L12 | X18 | MEP | 0.7 | n.d. | 1.4 | 1.8 |
| L1 | X19 | MEP | 0.5 | n.d. | 4.5 | 5.4 |
| L10 | X19 | MEP | 0.7 | n.d. | 14.4 | 14.8 |
| L12 | X19 | MEP | 0.5 | n.d. | 6.9 | 6.6 |
| L1 | X20 | MEP | 2.8 | n.d. | 12.9 | 14.3 |
| L10 | X20 | MEP | 2.2 | n.d. | 18.2 | 16.9 |
| L12 | X20 | MEP | 1.5 | n.d. | 4.6 | 4.2 |

*Stability studies performed in buffers as described in example C6

1-20. (canceled)

21. A hydroxyalkyl starch (HAS) derivative of formula (I) 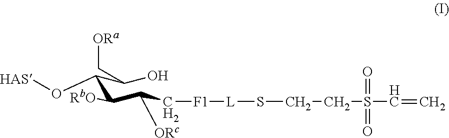
wherein
F1 is a functional group comprising the group —NR′—, with R′ being H or alkyl;
L is a spacer bridging F1 and S;
HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are, independently of each other, hydrogen or an alkyl group having from 1 to 4 carbon atoms, m is 2 to 4, wherein R¹ and R² are the same or different from each other in the m groups CR¹R²; n is from 0 to 6.

22. The HAS derivative of claim 21, wherein the HAS is hydroxyethyl starch (HES),
R¹, R², R³, and R⁴ are hydrogen,
m is 2;
n is 0 to 4.

23. The HAS derivative of claim 21, wherein F1 is selected from the group consisting of —NH—, —NH—NH—, —NH—NH—C(═O)—, and —NH—O—.

24. The HAS derivative of claim 21, wherein the spacer L comprises the moiety —(C(L′L″))_{q}- with L′ and L″ in each repeating unit CL′L″ with L′ and L″ in each repeating unit —C(L′L″)- being, independently of each other, selected from the group consisting of H, alkyl, aryl, alkenyl, alkynyl, hydroxyl, fluorine, alkylcarbonyloxy, arylcarbonyloxy, alkoxycarbonyloxy, aryloxycarbonyloxy, amide, carboxyl, alkoxycarbonyl, aminocarbonyl, alkylaminocarbonyl, dialkylaminocarbonyl, alkoxy, phosphate, phosphonato, phosphinato, tertiary amino, acylamino, including alkylcarbonylamino, arylcarbonylamino, carbamoyl, ureido, nitro, alkylthio, arylthio, sulfate, alkylsulfinyl, sulfonate, sulfonamido, trifluoromethyl, cyano, azido, carboxymethylcarbamoyl, cycloalkyl, heterocycloalkyl, alkylaryl, arylalkyl, and heteroaryl, wherein the groups L′ and L″ in each repeating unit may be the same or may differ from each other, with q being in the range of from 1 to 20.

25. The HAS derivative of claim 21, wherein the spacer L is —CH₂—CH₂—.

26. The HAS derivative of claim 21, wherein Q is GLP-1 or GLP-2.

27. A hydroxyalkyl starch (HAS) derivative of formula (IV) 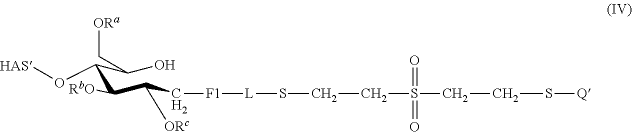
wherein
Q′ is the remainder of a thiol group comprising compound Q which is linked via the group —S— of the thiol group to the —CH₂ group;
F1 is a functional group comprising the group NR′—, with R′ being H or alkyl;
L is a spacer bridging F1 and S;
HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are, independently of each other, hydrogen or an alkyl group having from 1 to 4 carbon atoms, m is 2 to 4, wherein R¹ and R² are the same or different from each other in the m groups CR¹R²; n is from 0 to 6.

28. The HAS derivative of claim 27, wherein Q is selected from the group consisting of peptides, polypeptides, proteins, enzymes, small molecule drugs, dyes, nucleosides, nucleotides, oligonucleotides, polynucleotides, nucleic acids, cells, viruses, liposomes, microparticles, micelles, and derivatives thereof.

29. The HAS derivative of claim 27, wherein the HAS is hydroxyethyl starch (HES),
R¹, R², R³, and R⁴ are hydrogen,
m is 2;
n is 0 to 4.

30. The HAS derivative of claim 27, wherein F1 is selected from the group consisting of —NH—, —NH—NH—, —NH—NH—C(═O)—, and —NH—O—.

31. The HAS derivative of claim 27, wherein the spacer L comprises the moiety —(C(L′L″))_{q}- with L′ and L″ in each repeating unit CL′L″ with L′ and L″ in each repeating unit —C(L′L″)- being, independently of each other, selected from the group consisting of H, alkyl, aryl, alkenyl, alkynyl, hydroxyl, fluorine, alkylcarbonyloxy, arylcarbonyloxy, alkoxycarbonyloxy, aryloxycarbonyloxy, amide, carboxyl, alkoxycarbonyl, aminocarbonyl, alkylaminocarbonyl, dialkylaminocarbonyl, alkoxy, phosphate, phosphonato, phosphinato, tertiary amino, acylamino, including alkylcarbonylamino, arylcarbonylamino, carbamoyl, ureido, nitro, alkylthio, arylthio, sulfate, alkylsulfinyl, sulfonate, sulfonamido, trifluoromethyl, cyano, azido, carboxymethylcarbamoyl, cycloalkyl, heterocycloalkyl, alkylaryl, arylalkyl, and heteroaryl, wherein the groups L′ and L″ in each repeating unit may be the same or may differ from each other, with q being in the range of from 1 to 20.

32. The HAS derivative of claim 27, wherein the spacer L is —CH₂—CH₂—.

33. The HAS derivative of claim 27, wherein Q is GLP-1 or GLP-2.

34. A method for the preparation of a hydroxyalkyl starch derivative comprising
(i) reacting hydroxyalkyl starch (HAS) of formula (Ia) 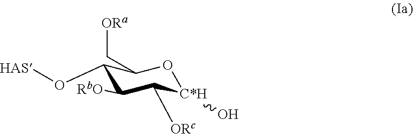
via carbon atom C* of the reducing end of the HAS with the functional group M of a crosslinking compound according to formula (II)
M-L-S-T (II)
wherein
M comprises the group —NHR′, with R′ being H or alkyl;
L is a spacer bridging M and S;
T is H or a thiol protecting group PG;
HAS′ is the remainder of the HAS molecule, R^{b} and R^{c} are —[(CR¹R²)ₘO]ₙ—H and are the same or different from each other; R^{a} is —[(CR¹R²)ₘO]ₙ—H with HAS′ being the remainder of the hydroxyalkyl starch molecule, or R^{a} is HAS″ with HAS′ and HAS″ together being the remainder of the hydroxyalkyl starch molecule; R¹ and R² are, independently of each other, hydrogen or an alkyl group having from 1 to 4 carbon atoms, m is 2 to 4, wherein R¹ and R² are the same or different from each other in the m groups CR¹R²; n is from 0 to 6,
thereby obtaining a HAS derivative of formula (Ib) 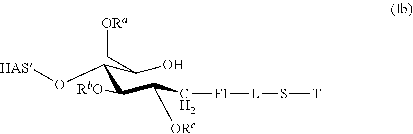
wherein —CH₂—F1- is the moiety resulting from the reaction of the group M with the HAS via the carbon atom C* of the reducing end, and F1 is a functional group comprising the group —NR′—; optionally removing PG in case T is PG to give T=H;
(ii) reacting the HAS derivative of formula (Ib) with a crosslinking compound of formula (III) 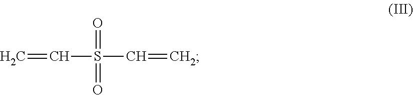
thereby obtaining a HAS derivative of formula (I)

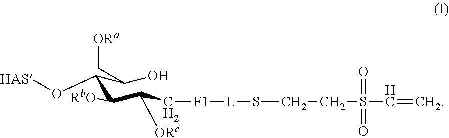

35. The method of claim 34, wherein T is a thiol protecting group PG, and wherein step (i) further comprises removing PG from the HAS derivative (Ib).

36. The method of claim 34, wherein M is selected from the group consisting of H₂N—, H₂N—NH—, H₂N—NH—C(═O)—, and H₂N—O—.

37. The method of claim 34, wherein the reacting according to step (i) is carried out under reductive amination conditions at a temperature in the range of from 5° C. to 100° C. and in a solvent selected from the group consisting of DMSO, DMF, NMP, DMA, formamide, water, reaction buffers, and mixtures thereof, and wherein the reacting according to step (ii) is carried out at a temperature in the range of from 0° C. to 50° C. and in a solvent selected from the group consisting of DMSO, DMF, NMP, DMA, formamide, water, reaction buffers, and mixtures thereof at a pH in the range of from 2 to 10.

38. The method of claim 34, further comprising
(iii) reacting the HAS derivative of formula (I) via the group —CH═CH₂ with an —SH group of a thiol group comprising compound Q, thereby forming a HAS derivative of formula (IV) 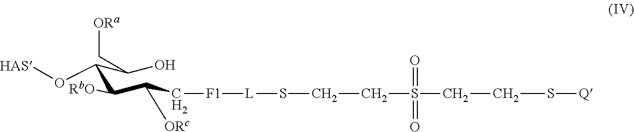
wherein
Q′ is the remainder of the thiol group comprising compound Q which is linked via the group —S— of the thiol group to the —CH₂ group, wherein Q is selected from the group consisting of peptides, polypeptides, proteins, enzymes, small molecule drugs, dyes, nucleosides, nucleotides, oligonucleotides, polynucleotides, nucleic acids, cells, viruses, liposomes, microparticles, micelles, and derivatives thereof.

39. The method of claim 38, wherein Q is a peptide, polypeptide, protein, or derivative thereof, and wherein the reacting according to step (iii) is carried out at a temperature in the range of from 0° C. to 50° C. and in a solvent selected from the group consisting of water, reaction buffers, DMSO, DMF, DMA, NMP, formamide, and mixtures of two or more thereof.

40. A method of using a HAS derivative as claimed in claim 21 as reactant for coupling to a thiol group comprising compound Q.
